(19) United States
(12) Patent Application Publication
(10) Pub. No.: US 2010/0105035 A1
(43) Pub. Date: Apr. 29, 2010

(54) ELECTROLUMINESCENT-BASED FLUORESCENCE DETECTION DEVICE

(76) Inventors: Syed Anwar Hashsham, Okemos, MI (US); James M. Tiedje, Lansing, MI (US); Erdogan Gulari, Ann Arbor, MI (US); Dieter Tourlousse, East Lansing, MI (US); Robert Stedtfeld, Lansing, MI (US); Farhan Ahmad, East Lansing, MI (US); Gregoire Seyrig, Lansing, MI (US); Onnop Srivannavit, Ann Arbor, MI (US)

Correspondence Address: MEDLEN & CARROLL, LLP, 101 HOWARD STREET, SUITE 350, SAN FRANCISCO, CA, 94105, US

(21) Appl. No.: 12/312,686

(22) PCT Filed: Nov. 21, 2007
(86) PCT No.: PCT/US07/24290

§ 371 Date: Dec. 10, 2009

Related U.S. Application Data
(60) Provisional application No. 60/860,702, filed on Nov. 22, 2006.

(51) Int. Cl. C12Q 1/68; G01J 1/58; C40B 60/12; C12M 1/34; C12M 1/40; C12Q 1/04; C12P 19/34

(52) U.S. Cl. 435/6; 250/458.1; 506/39; 435/288.7; 250/459.1; 435/34; 435/91.2

(57) ABSTRACT

The present invention provides compositions providing and methods using fluorescence detection device, comprising an electroluminescent light (EL) source, for measuring fluorescence in biological samples. In particularly preferred embodiments, the present invention provides an economical, battery powered and Hand-held device for detecting fluorescent light emitted from reporter molecules incorporated into DNA, RNA, proteins or other biological samples, such as a fluorescence emitting biological sample on a microarray chip. Further, a real-time hand-held PCR Analyzer device comprising an EL light source for measuring fluorescence emissions from amplified DNA is provided.

This invention was made with government support from the National Institutes of Health; grant numbers 1R01RR018625-01, 5R01RR018625-02, 1 R01 RR018625-03 and 5R01RR018625-03. The United States Government has certain rights in the invention.

### FIELD OF THE INVENTION

The present invention provides compositions providing and methods using a fluorescence detection device, comprising an electroluminescent light (EL) source, for measuring fluorescence in biological samples. In particularly preferred embodiments, the present invention provides a device comprising an electroluminescent (EL) film, for providing an economical, battery powered and hand-held device for detecting fluorescent light emitted from reporter molecules incorporated into DNA, RNA, proteins or other biological samples, such as a fluorescence emitting biological sample on a microarray chip. Further, a real-time hand-held PCR analyzer device comprising an EL light source for measuring fluorescence emissions from amplified DNA is provided.

### BACKGROUND OF THE INVENTION

Laser-based fluorescence detectors are currently the workhorses of diagnostic and research laboratories. These detectors typically use lasers, e.g. argon-ion, for providing stationary UV transilluminators and UV stations for detecting optical and/or fluorescent light emissions from a wide variety of colored molecules and/or florescent molecules marking biological samples. However, these detectors have a limited range of types of fluorescent emissions while operators must protect against exposure to harmful laser emissions.

Recently, white light transilluminators based upon electroluminescent light sources, similar to those light sources used in LED backlighting, were provided commercially for detecting certain types of fluorescence in conjunction with UV transilluminators or as stand alone bench top devices. However, although these detectors are safer when based upon electroluminescent light, these stations remain large, stationary, expensive, have a limited range for detecting types of optical emissions, specifically, fluorescence emissions, and do not measure real-time fluorescence emissions.

Therefore, there is a need for new types of fluorescence detectors to overcome or substantially ameliorate at least one of the above disadvantages.

### SUMMARY OF THE INVENTION

The present invention provides compositions providing and methods using a fluorescence detection device, comprising an electroluminescent light (EL) source, for measuring fluorescence in biological samples. In particularly preferred embodiments, the present invention provides an economical, battery powered and hand-held device for detecting fluorescent light emitted from reporter molecules incorporated into DNA, RNA, proteins or other biological samples, such as a fluorescence emitting biological sample on a microarray chip. Further, a real-time hand-held PCR Analyzer device comprising an EL light source for measuring fluorescence emissions from amplified DNA is provided.

For example, the present invention provides fluorescence detection devices comprising an electroluminescent light (EL) source that provide static and/or real-time fluorescent read-outs in a number of formats including visual and digital. In further examples, the present invention provides fluorescence detection devices comprising an electroluminescent light (EL) source that provides PCR assay capabilities, such as thermal cycling assays, and isothermal amplification assays, computational capabilities for data read-outs, and read-out capabilities in a number of formats including visual and digital.

It is not intended that the present invention be limited by the nature of the reactions carried out in the electroluminescent fluorescence detection device. Reactions include, but are not limited to, chemical and biological reactions. Biological reactions include, but are not limited to mRNA transcription, nucleic acid amplification, DNA amplification, cDNA amplification, sequencing, and the like. It is also not intended that the invention be limited by the particular purpose for carrying out the biological reactions. In one diagnostic application, it may be desirable to simply detect the presence or absence of a particular pathogen. In another diagnostic application, it may be desirable to simply detect the presence or absence of specific allelic variants of pathogens in a clinical sample. For example, different species or subspecies of bacteria may have different susceptibilities to antibiotics; rapid identification of the specific species or subspecies present aids diagnosis and allows initiation of appropriate treatment.

The present invention provides a device, comprising, a) an electroluminescent light source, b) an excitation filter, c) a biological sample holder, and d) an emission filter, wherein said biological sample holder, is disposed between said excitation filter and said emission filter and said electroluminescent light source is adjacent to said excitation filter so that light produced by said electroluminescent light source passes through said excitation filter to illuminate said biological sample holder. The present invention is not limited to a particular electroluminescent light source. Indeed, a variety of electroluminescent light sources may be incorporated, including, but not limited to a blue, blue-green and green electroluminescent film. Indeed, a variety of emission filters and excitation filters may be incorporated, including, but not limited to Super Gel filters, in any case, the emission filter and excitation filter should be optically compatible with the electroluminescent light source and a target fluorescent molecule. The present invention is not limited to a particular biological sample holder. Indeed, a variety of biological sample holders may be used, including, but not limited to a biological sample holder of the present invention. In one embodiment, the biological sample holder is compatible with a PCR chip. In one embodiment, the biological sample holder is compatible with a microarray chip. In one embodiment, the biological sample holder is stationary. In one embodiment, the biological sample holder is mobile.

In one embodiment, the device further comprises an optical signal detector positioned to detect optical signals from a biological sample contained in said biological sample holder. Indeed, a variety of optical signal detector types may be incorporated, including, but not limited to an optical signal detector is selected from the group consisting of a charge-coupled device (CCD) and complimentary metal-oxide semiconductor (CMOS) image chip. In one embodiment, the device comprises an external case enclosing said electroluminescent light source, excitation filter, biological sample holder, and emission filter. The present invention is not limited to a particular external case. Indeed, a variety of cases are contemplated, including but not limited to a hard case or a soft case. The present invention is limited to a particular size. In one embodiment, the device weighs 2 pounds or less. In one embodiment, the device weighs 1 pound or less. In one embodiment, the diameter of the device is less than 11×3.5×7 inches. In one embodiment, the device further comprises an electrical power source. The present invention is not limited to a particular electrical power source. Indeed, a variety of electrical power sources are contemplated, including but not limited to an AC power source and/or a DC power source electrically connected to said electroluminescent light source. In one embodiment, the device further comprises a battery power source electrically connected to said electroluminescent light source. The present invention is not limited to a particular battery power source. Indeed, a variety of battery power sources are contemplated, including but not limited to an internal battery power source or an external battery power source. In one embodiment, the device further comprises a peripheral. The present invention is not limited to any particular peripheral. Indeed, a variety of peripherals are contemplated including but not limited to an external USB hard drive and/or an electrically connected wireless communication chip. In a further embodiment, the biological sample holder comprises an optically compatible assay. The present invention is not limited to a particular assay. Indeed, a variety of biological assays are contemplated, including but not limited to microarray chip or a PCR chip. In a further embodiment, the assay comprises a biological sample. In one embodiment, the microarray chip comprises a biological sample. In one embodiment, the PCR chip comprises a biological sample. The present invention is not limited to a particular biological sample. Indeed, a variety of biological samples are contemplated, including but not limited to DNA, RNA and protein. In yet a further embodiment, the biological sample is labeled with a fluorescent compound. The present invention is not limited to a particular fluorescent compound. Indeed, a variety of fluorescent compounds are contemplated, including but not limited to SYBR™ Brillant Green, SYBR™ Green I, SYBR™ Green II, SYBR™ gold, SYBR™ safe, EvaGreen™, a green fluorescent protein (GFP), fluorescein, ethidium bromide (EtBr), thiazole orange (TO), oxazole yellow (YO), thiarole orange (TOTO), oxazole yellow homodimer (YOYO), oxazole yellow homodimer (YOYO-1), SYPRO® Ruby, SYPRO® Orange, Coomassie Flour™ Orange stains, and derivatives thereof.

The present invention contemplates a system, comprising, a) an electroluminescent light source, b) an excitation filter, c) a biological sample, d) an emission filter, and e) an optical signal detector, wherein said biological sample is disposed between said excitation filter and said emission filter and said electroluminescent light source is adjacent to said excitation filter so that light produced by said electroluminescent light source passes through said excitation filter to illuminate said biological sample, and emitted light from said biological sample passes through said emission filter so that it is detectable by said optical signal detector.

The present invention is not limited to a particular electroluminescent light source. Indeed, a variety of electroluminescent light sources may be incorporated, including, but not limited to a blue, blue-green and green electroluminescent film. Indeed, a variety of emission filters and excitation filters may be incorporated, including, but not limited to Super Gel filters, in any case, the emission filter and excitation filter should be optically compatible with the electroluminescent light source and a target fluorescent molecule. The present invention is not limited to a particular biological sample holder. Indeed, a variety of biological sample holders may be used, including, but not limited to a biological sample holder of the present invention. In one embodiment, the biological sample holder is compatible with a PCR chip. In one embodiment, the biological sample holder is compatible with a microarray chip. In one embodiment, the biological sample holder is stationary. In one embodiment, the biological sample holder is mobile.

In one embodiment, the device further comprises an optical signal detector positioned to detect optical signals from a biological sample contained in said biological sample holder. Indeed, a variety of optical signal detector types may be incorporated, including, but not limited to an optical signal detector is selected from the group consisting of a charge-coupled device (CCD) and complimentary metal-oxide semiconductor (CMOS) image chip. In one embodiment, the device comprises an external case enclosing said electroluminescent light source, excitation filter, biological sample holder, and emission filter. The present invention is not limited to a particular external case. Indeed, a variety of cases are contemplated, including but not limited to a hard case or a soft case. The present invention is limited to a particular size. In one embodiment, the device weighs 2 pounds or less. In one embodiment, the device weighs 1 pound or less. In one embodiment, the diameter of the device is less than 11×3.5×7 inches. In one embodiment, the device further comprises an electrical power source. The present invention is not limited to a particular electrical power source. Indeed, a variety of electrical power sources are contemplated, including but not limited to an AC power source and/or a DC power source electrically connected to said electroluminescent light source. In one embodiment, the device further comprises a battery power source electrically connected to said electroluminescent light source. The present invention is not limited to a particular battery power source. Indeed, a variety of battery power sources are contemplated, including but not limited to an internal battery power source or an external battery power source. In one embodiment, the device further comprises a peripheral. The present invention is not limited to any particular peripheral. Indeed, a variety of peripherals are contemplated including but not limited to an external USB hard drive and/or an electrically connected wireless communication chip. In a further embodiment, the biological sample holder comprises an optically compatible assay. The present invention is not limited to a particular assay. Indeed, a variety of biological assays are contemplated, including but not limited to microarray chip or a PCR chip. In a further embodiment, the assay comprises a biological sample. In one embodiment, the microarray chip comprises a biological sample. In one embodiment, the PCR chip comprises a biological sample. The present invention is not limited to a particular biological sample. Indeed, a variety of biological samples are contemplated, including but not limited to DNA, RNA and protein. In yet a further embodiment, the biological sample is labeled with a fluorescent compound. The present invention is not limited to a particular fluorescent compound. Indeed, a variety of fluorescent compounds are contemplated, including but not limited to SYBR™ Brillant Green, SYBR™ Green I, SYBR™ Green II, SYBR™ gold, SYBR™ safe, EvaGreen™, a green fluorescent protein (GFP), fluorescein, ethidium bromide (EtBr), thiazole orange (TO), oxazole yellow (YO), thiarole orange (TOTO), oxazole yellow homodimer (YOYO), oxazole yellow homodimer (YOYO-1), SYPRO® Ruby, SYPRO® Orange, Coomassie Fluor™ Orange stains, and derivatives thereof.

The present invention provides a method of detecting emitted fluorescent light, comprising: a) providing an electroluminescent light source and a biological sample labeled with a fluorescent compound; b) illuminating said biological sample with said electroluminescent light source; and c) detecting light emitted from said biological sample. The present invention is not limited to a particular electroluminescent light source. Indeed, a variety of electroluminescent light sources may be incorporated, including, but not limited to a blue, blue-green and green electroluminescent film. The present invention is not limited to a particular biological sample. Indeed, a variety of biological samples are contemplated, including but not limited to DNA, RNA and protein. In yet a further embodiment, the biological sample is labeled with a fluorescent compound. The present invention is not limited to a particular fluorescent compound. Indeed, a variety of fluorescent compounds are contemplated, including but not limited to SYBR™ Brillant Green, SYBR™ Green I, SYBR™ Green II, SYBR™ gold, SYBR™ safe, EvaGreen™, a green fluorescent protein (GFP), fluorescein, ethidium bromide (EtBr), thiazole orange (TO), oxazole yellow (YO), thiarole orange (TOTO), oxazole yellow homodimer (YOYO), oxazole yellow homodimer (YOYO-1), SYPRO® Ruby, SYPRO® Orange, Coomassie Fluor™ Orange stains, and derivatives thereof. In a further embodiment, the biological sample is contained in a sample chamber of a microarray chip. In a further embodiment, the biological sample is provided on a microarray. In a further embodiment, the biological sample is contained in a sample chamber of a PCR chip. The invention is not limited to the type of detecting. Indeed, a variety of types of detecting are contemplated including but not limited to a charge-coupled device (CCD) and complimentary metal-oxide semiconductor (CMOS) image chip. In some preferred embodiments, the EL-devices and methods do not utilize an light source, such as a UV light source, in addition to the EL source.

The present invention provides a device, comprising, a) an electroluminescent illumination light source, wherein said electroluminescent light source comprises an electroluminescent film, and b) a biological sample chamber. In some embodiments, the electroluminescent film comprises at least one layer of indium-tin oxide. In some embodiments, the layer of indium-tin oxide is optically transparent. In some embodiments, the layer of indium-tin oxide is provided as a layer selected from the group consisting of a sputter deposition, an electron beam evaporation deposition, and a physical vapor deposition. In some embodiments, the electroluminescent film comprises at least one layer selected from the group consisting of a polymer, a metal foil, electroluminescent phosphor ink, conductive ink, electroluminescent phosphor layer, a transparent polyester film, and a dielectric layer. In some embodiments, the biological sample chamber is optically transparent. In some embodiments, the biological sample chamber comprises a chip, wherein said chip is optically transparent. In some embodiments, the chip selected from the group consisting of a microarray chip, a multichannel chip, and an on-chip DNA amplification chip. In some embodiments, the chip comprises a biological sample. In some embodiments, the biological sample comprises a fluorescent compound. In some embodiments, the device further comprises at least one component selected from the group consisting of excitation filter, emission filter, optical signal detector, thin-film heater, software, a liquid crystal display, a Universal Serial Bus port, and an external case.

The present invention provides a method of detecting emitted fluorescent light, comprising: a) providing, i) an electroluminescent illumination light source, wherein said electroluminescent light source comprises an electroluminescent film, and ii) a biological sample, wherein said biological sample comprises a fluorescent compound, b) illuminating said biological sample with said electroluminescent illumination light source; and c) detecting an optical signal emitted from said fluorescent compound. In some embodiments, the biological sample is selected from the group consisting of DNA, RNA and protein. In some embodiments, the biological sample comprises DNA. In some embodiments, the method further comprises amplifying said DNA prior to detecting an optical signal. In some embodiments, the amplifying DNA is selected from the group consisting of an isothermal amplification and a polymerase chain reaction amplification. In some embodiments, the biological sample comprises a fluorescent compound, wherein said fluorescent compound is selected from the group consisting of SYBR™ Brillant Green, SYBR™ Green I, SYBR™ Green II, SYBR™ gold, SYBR™ safe, EvaGreen™, a green fluorescent protein (GFP), fluorescein, ethidium bromide (EtBr), thiazole orange (TO), oxazole yellow (YO), thiarole orange (TOTO), oxazole yellow homodimer (YOYO), oxazole yellow homodimer (YOYO-1), SYPRO Ruby, SYPRO® Orange, Coomassie Fluor™ Orange stains, and derivatives thereof. In some embodiments, the biological sample comprises a water sample. In some embodiments, the detecting comprises a real-time measurement, a positive/negative answer, and pathogen identification.

### DESCRIPTION OF THE FIGURES

FIG. 1 shows exemplary types of commercially available electroluminescence (EL) products.

FIG. 2 shows an exemplary schematic diagram of an electroluminescent (EL) unit for emitting light. Please note that elements in this diagram are not drawn to scale.

FIG. 3 shows a) one exemplary schematic diagram of an EL-based fluorescence detector of the present invention and actual photographs of EL-film without an electrical current (off) and with an electrical current (on), with actual illumination results b) a black and white fluorescence CCD camera image and c) a colored photographic image. EL illuminated biological material was labeled with SYBR Green. Please note that elements in this diagram are not drawn to scale.

FIG. 4 shows one exemplary schematic of EL-based hand-held fluorescence detector of the present invention. A) Internal front view and B) Internal side view. Please note that elements in this diagram are not drawn to scale.

FIG. 5 shows an exemplary schematic of internal CMOS camera module and LCD external display for EL-based florescence detection. Please note that elements in this diagram are not drawn to scale.

FIG. 6 shows an exemplary A) external image of an EL-based hand-held fluorescence detector of the present invention and B) chip for insertion into hand-held detector of the present invention (note fingers in image for scale). Please note that elements in this diagram are not drawn to scale.

FIG. 7 shows an exemplary schematic diagram with actual examples of elements of the image path of an EL-Based hand-held pathogen analyzer of the present invention. Please note that elements in this diagram are not drawn to scale.

FIG. 8 shows one exemplary schematic of an EL-based PCR chip analyzer components A) CCD camera and SYBR excitation and emission filters, B) transparent integrated heater and Peltier cooling for low power consumption, lightweight, and MEMS-based construction, and C) Electroluminescent Film (for example, 0.2 mm thick) for an illumunination source with low power consumption, low heat generation and lightweight. Please note that elements in this diagram are not drawn to scale.

FIG. 9 shows exemplary heating components for use in ELF devices of the present inventions.

FIG. 10 shows an exemplary computer-aided design (CAD) schematic of a PCR chip for on-chip PCR analysis for use within an EL-Based Pathogen Analyzer of the present invention. Please note that elements in this diagram are not drawn to scale.

FIG. 11 shows an exemplary schematic of on-chip primers A) prior to amplification and B) during the first heat cycle. Please note that elements in this diagram are not drawn to scale.

FIG. 12 shows an exemplary estimated cost for providing data using an EL-based hand-held pathogen analyzer of the present inventions.

FIG. 13 shows an exemplary comparison of cost per sample between PCR chip & EL-based bench-top and PCR Chip & EL-based hand-held pathogen analyzer and commercially available devices.

FIG. 14 shows an exemplary graph comparison of cost per sample between PCR chip & EL-based bench-top and PCR chip & EL-based hand-held pathogen analyzer and commercially available devices.

FIG. 15 shows an exemplary semi-log scale graph comparison of cost per sample between PCR chip & EL-based bench-Top and PCR Chip & EL-based hand-held pathogen analyzer and commercially available devices.

FIG. 16 shows an exemplary comparison of cost estimates between a PCR Chip & EL-based hand-held pathogen analyzer of the present invention to commercially available microarrays/chips/samples and their corresponding analytical devices.

FIG. 17 shows exemplary units of a Handheld PCR system of the present inventions including major units associated with various tasks.

FIG. 18 shows an exemplary schematic of components contemplated for a hand-held real-time PCR device. Components along the top focus on sample processing while lower right corner is focused on amplification strategies. Boxes on lower left indicate the electronics and printed circuit board.

FIG. 19 shows an exemplary MicroPCR chip designs focusing on sealing, primer dispensing, and sample placement strategies under evaluation for use in a hand-held real time PCR device of the present inventions (A) (B) (C) Serpentine chip, please note that the solid base would need to be replaced with an optically transparent base for actual use in a real time PCR device of the present invention.

FIG. 20 shows an exemplary confirmation of amplification in a serpentine PCR chip demonstrating reaction products obtained from a nonleaking chip (a) microfulidic channel, (b) PCR product detectable after the 15^{th} cycle, and (c) demonstration of success obtaining the expected size PCR product by routine gel electrophoresis.

FIG. 21 shows exemplary the stability of exemplary freeze-dried PCR reagents (A) Optimization of trehalose concentration for freeze-dried Taq Polymerase and (B) Stability of freeze-dried PCR reagents with 15% Trehalose.

FIG. 22 shows an exemplary microfluidic DNA biochip with recirculation capabilities: (a) a chip approximately 1 cm2, (b) a close-up view of microlfuidic channels and a portion of the approximately 8,000 reactors on the chip, (c) a close-up view of 6 reactors, each with 50 m diameter, (d) signal to noise ratio for 5 genes belonging to one of the 20 organisms that were tested on the chip, and (e) laser scanned signal intensities for part of the chip. (f) A design proposing to cycle the microPCR chip instead of the Peltier units and including an imaging station for a real time PCR assay.

FIG. 23 shows an exemplary shows the complete setup of temperature measurement and control unit. Left panel shows the DAQ from National Instruments (suppliers of LabView) and right panel shows initial effort to calculate the rate of heating of a doped chip.

FIG. 24 shows an exemplary A) Circuit of temperature measurement unit and B) Complete circuit of temperature measurement and controller unit.

FIG. 25 shows an exemplary A) LABVIEW code for temperature measurement and control and B) Front Panel of LABVIEW Thermal Cycling Program.

FIG. 26 shows an exemplary LabView Program configuration for CCD camera image acquisition A) Labview code for Image Acquisition and B) Front Panel of Labview code written for Image Acquisition.

FIG. 27 shows A and B) a microfluidic chip known to detect influenza virus and (c-f) an exemplary micro-PCR device with integrated heaters. Due to very small reagent volume, the rate of heating can be as high as 165° C. per second reducing the time to PCR from hours to less than 6 minutes.

FIG. 28 shows exemplary components for devices of the present inventions that are commercially available including miniature pumps (a and b) for moving ul volumes, a fan (c), a laser for breaking cells (d) minicontrollers for controlling the components in devices of the present inventions, such as Texas Instrument's eZ430 microcontroller and development tool (e) cicuit boards and and peripherals, such as a Fingertip4 printed circuit board and peripherals from In-Hand electronics, and (f) an exemplary image of an external case for a hand-held real time PCR device of the present inventions.

FIG. 29 shows an exemplary highly parallel sequencing on a wafer.

FIG. 30 shows exemplary results from a helicase-dependent isothermal amplification.

FIG. 31 shows an exemplary analysis of literature for static, integrated heater, and Flow-through microPCR Chips: A) typical increasing trend of PCR time with the inverse of flow rate per unit cross sectional area of channel in continuous flow PCR systems B) A comparison of PCR time for integrated heaters (red bars) vs non-integrated heaters (blue bars) in a static PCR system.

FIG. 32 shows an exemplary analysis of literature for static, integrated heater, and Flow-through microPCR Chips: A) An inverse trend between the heating rate of heaters (integrated and non-integrated) and total PCR time for static PCR systems. Thermal mass of heaters for four studies has been shown with arrows. The decreasing thermal mass of heaters leads to increase the heating rate and decrease the amplification time B) A typical increasing trend of DNA amplification time with increasing thermal mass of integrated heaters in a static PCR system.

### DEFINITIONS

To facilitate an understanding of the present invention, a number of terms and phrases are defined below:

The use of the article “a” or “an” is intended to include one or more. As used in this application, the singular form “a,” “an,” and “the” include plural references unless the context clearly dictates otherwise. For example, the term “an agent” includes a plurality of agents, including mixtures thereof.

As used herein, “electroluminescence” or “EL” refer to a direct conversion of electrical energy into light by a luminescent material such as a light emitting phosphor.

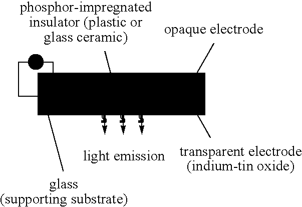

As used herein, “ACTFEL” and “alternating current thin film electroluminescence” refers to emitted light following exposure to an electrical current.

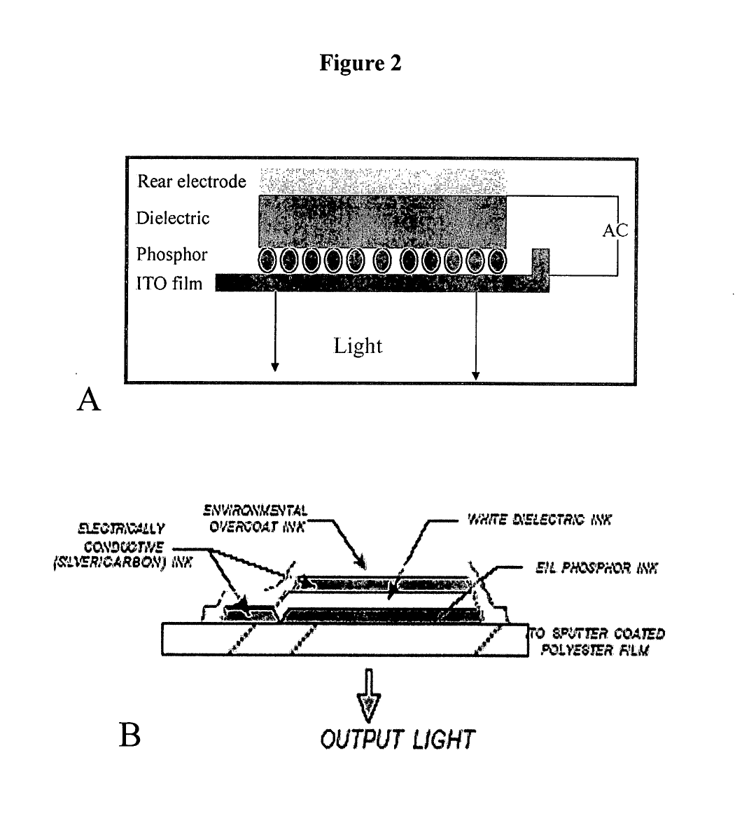

As used herein, “electroluminescent sheet” and “electroluminescent film” and “ELM” and “electroluminescent panel” and “electroluminescent wire” and “electroluminescent lamp” and “EL lamp” refer to a type of capacitor comprising a thin layer of light emitting phosphor located between two electrodes, wherein in one example, an electroluminescent film comprises a first electrode, wherein said electrode is opaque and a second electrode, wherein said second electrode is translucent in order to allow light to escape. In another example, an electroluminescent sheet comprises a first transparent electrode and a second transparent electrode, for example, an electrode comprising ITO. Further examples of electroluminescent film comprise at least one layer selected from the group consisting of a polymer, a metal foil, electroluminescent phosphor ink, conductive ink, electroluminescent phosphor layer, a transparent polyester film, and a dielectric layer, see, NOVATECH™ Blue/Green output EL lamps, Novatech, Chino, Calif., U.S. Patent Application No. 20030003837, herein incorporated by reference, and FIG. 2.

As used herein, “capacitor” refers to an electrical device that can store energy in the electric field between a pair of conductors or ‘plates,’ such as electrodes. In one embodiment of the present invention, a specialized capacitor is an electroluminescent film, for example, see, FIG. 2.

As used herein, “electrode” refers to a plate of the capacitor, for example, a capacitor such as an electroluminescent film. When use in reference to ELF, a capacitor may comprise one back electrode, wherein a “back electrode” is the electrode furthest away from a biological sample, for example, an electrode comprising silver, and one front electrode, wherein a “front electrode” is the electrode nearest a biological sample, such an electrode comprising as transparent ITO film, for examples, see, Noach Appl. Phys. Lett. 69(24):3650- 3652; herein incorporated by reference. For the purposes of the present invention, “transparent electrode” refers to an electrode “transparent to light,” such as a transparent ITO layer.

As used herein, “indium-tin oxide film” or “ITO film” refers to a protective optical coating that is transparent and conductive to light, for example, a thin film EL, such that a composition of Indium Tin Oxide (In203:Sn02) is a layer of indium oxide that has been doped with tin.

As used herein, “layer” in reference to a compound, refers to a deposition of the compound by methods such as sputter deposition, an electron beam evaporation deposition, and a physical vapor deposition.

As used herein, “emitting layer” refers to a layer comprising a substance that upon electrical stimulation will emit light, such as a phosphor in a phosphor layer of an ELF.

As used herein, “phosphor” refers to a substance that exhibits the phenomenon of phosphorescence, either natural, for example, a transition metal compound or rare earth compound, or synthetic, for example, a suitable host material, to which an activator is added such as a copper-activated zinc sulfide and the silver-activated zinc sulfide (zinc sulfide silver).

As used herein, “phosphor” in reference to a powder refers to a material such as zinc sulfide, doped with either copper or manganese to achieve a desired emission color when exposed to an electric field. For one example, when AC current (400-1600 Hz) is applied to a phosphor resulting in the emission of light, such that the phosphor chemical composition and associated dye pigments determine the brightness and color of the emitted light in combination with the strength of the applied current.

As used herein, “dielectric” refers to a substance, such as a solid, liquid, or gas, that is highly resistant to electric current n electric field polarizes the molecules of the dielectric, producing concentrations of charge on its surfaces that create an electric field opposed (for example, antiparallel) to that of the capacitor. Thus, a given amount of charge produces a weaker field between the plates than it would without the dielectric, which reduces the electric potential.

As used herein, “dielectric layer” refers to an insulating layer, for example, a layer that serves to even out the electric field across the phosphor layer and prevent a short circuit.

As used herein, “filter” refers to a device or coating that preferentially allows light of characteristic spectra to pass through it (e.g., the selective transmission of light beams).

As used herein, “light” refers to electromagnetic radiation with a wavelength that is visible to the human eye (such as, visible light) or, in a technical or scientific context, electromagnetic radiation of any wavelength. As used herein, light comprises three basic dimensions of intensity, frequency and polarization.

As used herein, “intensity” or “amplitude” refers to a human perception of brightness of the light, and polarization (such as an angle of vibration).

As used herein, “frequency” refers to a number of oscillations (vibrations) in one second. Frequency f is the reciprocal of the time T taken to complete one cycle (the period), or 1/T. The frequency with which earth rotates is once per 24 hours. Frequency is usually expressed in units called hertz (Hz). Frequency is measured in terms “hertz” or “Hz” that refer to “oscillations per second” or “cycles per second such that “one hertz” or “1 Hz” is equal to one cycle per second, for example, “one kilohertz” or “kHz” is 1,000 Hz, and “one megahertz” or “MHz” is 1,000,000 Hz. Electromagnetic radiation is also measured in kiloHertz (kHz), megahertz (MHz) and gigahertz (GHz).

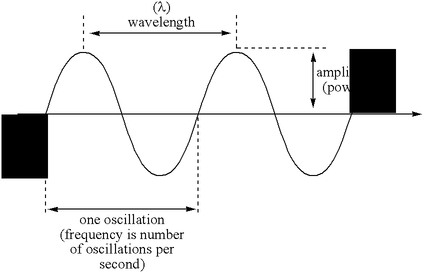

As used herein, the term “transducer device” refers to a device that is capable of converting a non-electrical phenomenon into electrical information, and transmitting the information to a device that interprets the electrical signal. Such devices can include, but are not limited to, devices that use photometry, fluorometry, and chemiluminescence; fiber optics and direct optical sensing (e.g., grating coupler); surface plasmon resonance; potentiometric and amperometric electrodes; field effect transistors; piezoelectric sensing; and surface acoustic wave.

As used herein, the term “optical transparency” refers to the property of matter whereby the matter is capable of transmitting light such that the light can be observed by visual light detectors (e.g., eyes and detection equipment).

As used herein, the term “film” refers to any substance capable of coating at least a portion of a substrate surface and immobilizing capture particles. Examples of materials used to make such films include, but are not limited to, agarose, acrylamide, SEPHADEX, proteins (e.g., bovine serum albumin (BSA), polylysine, collagen, etc.), hydrogels (e.g., polyethylene oxide, polyvinyl alcohol, polyhydroxyl butylate, etc.), film forming latexes (e.g., methyl and ethyl aerylates, vinylidine chloride, and copolymers thereof), or mixtures thereof In certain embodiments, films include additional material such as plasticizers (e.g., polyethylene glycol [PEG], detergents, etc.) to improve stability and/or performance of the film. In preferred embodiments, a film is a material that will react with the capture particles and present them in the same focal plane. In other preferred embodiments, a film is pre-activated with cross-linking groups such as aldehydes, or groups added after the film has been formed.

As used herein, “optical signal” refers to any energy (e.g., photodetectable energy) emitted from a sample (e.g., produced from a microarray that has one or more optically excited [i.e., by electromagnetic radiation] molecules bound to its surface).

As used herein, “filter” refers to a device or coating that preferentially allows light of characteristic spectra to pass through it (e.g., the selective transmission of light beams). “Polychromatic” and “broadband” as used herein, refer to a plurality of electromagnetic wavelengths emitted from a light source or sample whereas monochromatic refers to a single wavelength or a narrow range of wavelengths.

As used herein, “microarray” refers to a substrate with a plurality of molecules (e.g., nucleotides) bound to its surface. Microarrays, for example, are described generally in Schena, “Microarray Biochip Technology,” Eaton Publishing, Natick, Mass., 2000. Additionally, the term “patterned microarrays” refers to microarray substrates with a plurality of molecules non-randomly bound to its surface. As used herein, the term “optical detector” or “photodetector” refers to a device that generates an output signal when irradiated with optical energy. Thus, in its broadest sense the term optical detector system is taken to mean a device for converting energy from one form to another for the purpose of measurement of a physical quantity or for information transfer. Optical detectors include but are not limited to photomultipliers and photodiodes.

As used herein, the term “photomultiplier” or “photomultiplier tube” refers to optical detection components that convert incident photons into electrons via the photoelectric effect and secondary electron emission. The term photomultiplier tube is meant to include devices that contain separate dynodes for current multiplication as well as those devices that contain one or more channel electron multipliers.

As used herein, the term “photodiode” refers to a solid-state light detector type including, but not limited to PN, PIN, APD and CCD.

As used herein, the term “plate reader” in reference to a “detection device” refer to a device to detect the transmission of light through or reflection of light (i.e., polarized light or non-polarized light of specific wavelengths) from the surface of an assay, that for the purposes of the present invention the assay is a “microarray chip” and “PCR chip” or a “glass slide” comprising a PCR assay or a “plate” such as a 96-well plate and the like. For example, a microtiter plate reader measures transmittance, absorbance, or reflectance through, in, or from each well of a multitest device such as a microtiter testing plate (e.g., MicroPlate™ testing plates) or a miniaturized testing card (e.g., MicroCard™ miniaturized testing cards).

As used herein, “chip” in its broadest sense refers to a composition, such as a microarray chip, a multichanneled chip, a PCR chip, a semi-conductor chip, and the like.

As used herein, “thin layer” refers to a very thin deposition of a colloidal substance (such as a layer of phosphor, dielectric, silver, etc.) onto an ITO coated glass plate.

As used herein, “electronic power supply” refers to an electronic device that produces a particular DC voltage or current from a source of electricity such as a battery or wall outlet.

As used herein, “power adapter,” “transformer,” or “power supply” refer to an external power supply for laptop computers or portable or semi-portable electronic device As used herein, “AC adapter” refers to a rectifier to convert AC current to DC and a transformer to convert voltage from 120V down, for example, 15V or 12V or 9V.

As used herein, “power supply” refers to an electrical system that converts AC current from the wall outlet into the DC currents required by the computer circuitry.

As used herein, “external AC adaptor power brick” refers to an electronic device that produces AC current.

As used herein, “AC powered linear power supply” refers to a transformer to convert the voltage from the wall outlet to a lower voltage. An array of diodes called a diode bridge then rectifies the AC voltage to DC voltage. A low-pass filter smoothes out the voltage ripple that is left after the rectification. Finally a linear regulator converts the voltage to the desired output voltage, along with other possible features such as current limiting.

As used herein, “AC current” and “Alternating Current” and “AC” refers to a type of electrical current, the direction of which is reversed at regular intervals or cycles. In the United States, the standard is 120 reversals or 60 cycles per second.

As used herein, “DC current” and “Direct Current” and “DC” refers to a type of electricity transmission and distribution by which electricity flows in one direction through the conductor, usually relatively low voltage and high current. For typical 120 volt or 220-volt devices, DC must be converted to alternating current.

As used herein, “battery” refers to a device that stores chemical energy and makes it available in an electrical form. Batteries comprise electrochemical devices such as one or more galvanic cells, fuel cells or flow cell, examples include, lead acid, nickel cadmium, nickel metal hydride, lithium ion, lithium polymer, CMOS battery and the like.

As used herein, “CMOS battery” refers to a battery that maintains the time, date, hard disk and other configuration settings in the CMOS memory.

As used herein, “inverter ” or “rectifier” refers to a device that converts direct current electricity to alternating current either for stand-alone systems or to supply power to an electricity grid.

As used herein, “volt” and “V” refer to a unit of electrical force equal to that amount of electromotive force that will cause a steady current of one ampere to flow through a resistance of one ohm.

As used herein, “voltage ” refers to an amount of electromotive force, measured in volts, that exists between two points.

As used herein, “Ohm” refers to a measure of the electrical resistance of a material equal to the resistance of a circuit in which the potential difference of 1 volt produces a current of 1 ampere.

As used herein, “ampere” and “amp” refers to a unit of electrical current or rate of flow of electrons, such that one volt across one ohm of resistance causes a current flow of one ampere.

As used herein, “watt” or “W” refer to a measure of power, i.e., Volts multiplied by Amps=Watts. Watt may also refer to a rate of energy transfer equivalent to one ampere under an electrical pressure of one volt, for examples, one watt equals 1/746 horsepower, or one joule per second, i.e., voltage×current=amperage.

As used herein, “Charge-Coupled Device” and “CCD” refers to an electronic memory that records the intensity of light as a variable charge.

As used herein, “storage CCDs” refers to either a separate array (frame transfer) or individual photosites (interline transfer) coupled to each imaging photosite.

As used herein, “CMOS” or “Complementary-symmetry/metal-oxide semiconductor” refers to a both a particular style of digital circuitry design and the family of processes used to implement that circuitry on integrated circuits (chips).

As used herein, “CMOS IMAGE SENSOR” refers to a “CMOS-based chip” that records intensities of light as variable charges similar to a CCD chip. In one embodiment, as CMOS chip use less power than a CCD chip.

As used herein, “optical signal” refers to any energy (e.g., photodetectable energy) from a sample (e.g., produced from a microarray that has one or more optically excited [i.e., by electromagnetic radiation] molecules bound to its surface).

As used herein, “microarray” refers to a substrate with a plurality of molecules (e.g., nucleotides) bound to its surface. Microarrays, for example, are described generally in Schena, (2000)Microarray Biochip Technology, Eaton Publishing, Natick, Mass.; herein incorporated by reference. Additionally, the term “patterned microarrays” refers to microarray substrates with a plurality of molecules non-randomly bound to its surface.

As used herein, the terms “optical detector” and “photodetector” refers to a device that generates an output signal when exposed to optical energy. Thus, in its broadest sense, the term “optical detector system” refers devices for converting energy from one form to another for the purpose of measurement of a physical quantity and/or for information transfer. Optical detectors include but are not limited to photomultipliers and photodiodes, as well as fluorescence detectors.

As used herein, the term “TTL” stands for Transistor-Transistor Logic, a family of digital logic chips that comprise gates, flip/flops, counters etc. The family uses zero Volt and five Volt signals to represent logical “0” and “1” respectively.

As used herein, the term “dynamic range” refers to the range of input energy over which a detector and data acquisition system is useful. This range encompasses the lowest level signal that is distinguishable from noise to the highest level that can be detected without distortion or saturation.

As used herein, the term “noise” in its broadest sense refers to any undesired disturbances (i.e., signal not directly resulting from the intended detected event) within the frequency band of interest. One example of noise is the summation of unwanted or disturbing energy introduced into a system from man-made and natural sources. In another example, noise may distort a signal such that the information carried by the signal becomes degraded or less reliable.

As used herein, the term “signal-to-noise ratio” refers the ability to resolve true signal from the noise of a system. One example of computing a signal-to-noise ratio is by taking the ratio of levels of the desired signal to the level of noise present with the signal. In preferred embodiments of the present invention, phenomena affecting signal-to-noise ratio include, but are not limited to, detector noise, system noise, and background artifacts.

As used herein, the term “detector noise” refers to undesired disturbances (i.e., signal not directly resulting from the intended detected energy) that originate within the detector. Detector noise includes dark current noise and shot noise. Dark current noise in an optical detector system results from the various thermal emissions from the photodetector. Shot noise in an optical system is the product of the fundamental particle nature (i.e., Poisson-distributed energy fluctuations) of incident photons as they pass through the photodetector.

As used herein, the term “system noise” refers to undesired disturbances that originate within the system. System noise includes, but is not limited to noise contributions from signal amplifiers, electromagnetic noise that is inadvertently coupled into the signal path, and fluctuations in the power applied to certain components (e.g., a light source).

As used herein, the term “background” or “background artifacts” include signal components caused by undesired optical emissions from the microarray. These artifacts arise from a number of sources, including: non-specific hybridization, intrinsic fluorescence of the substrate and/or reagents, incompletely attenuated fluorescent excitation light, and stray ambient light. In some embodiments, the noise of an optical detector system is determined by measuring the noise of the background region and noise of the signal from the microarray feature.

As used herein, the term “processor” refers to a device that performs a set of steps according to a program (e.g., a digital computer). Processors, for example, include Central Processing Units (“CPUs”), electronic devices, and systems for receiving, transmitting, storing and/or manipulating digital data under programmed control.

As used herein, the terms “memory device,” and “computer memory” refer to any data storage device that is readable by a computer, including, but not limited to, random access memory, hard disks, magnetic (e.g., floppy) disks, zip disks, compact discs, DVDs, magnetic tape, and the like.

The term “gene” refers to a nucleic acid (e.g., DNA) sequence that comprises coding sequences necessary for the production of a polypeptide or precursor. It is intended that the term encompass polypeptides encoded by a full length coding sequence, as well as any portion of the coding sequence, so long as the desired activity and/or functional properties (e.g., enzymatic activity, ligand binding, etc.) of the full-length or fragmented polypeptide are retained. The term also encompasses the coding region of a structural gene and the sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as “5′ untranslated sequences.” The sequences that are located 3′ (i.e., “downstream”) of the coding region and that are present on the mRNA are referred to as “3′ untranslated sequences.” The term “gene” encompasses both cDNA and genomic forms of a gene. A genomic form of a genetic clone contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

Where “amino acid sequence” is recited herein to refer to an amino acid sequence of a naturally occurring protein molecule, “amino acid sequence” and like terms, such as “polypeptide” and “protein” is not meant to limit the amino acid sequence to the complete, native amino acid sequence associated with the recited protein molecule.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotide or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the terms “an oligonucleotide having a nucleotide sequence encoding a gene” and “polynucleotide having a nucleotide sequence encoding a gene,” means a nucleic acid sequence comprising the coding region of a gene or, in other words, the nucleic acid sequence that encodes a gene product. The coding region may be present in a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements include splicing signals, polyadenylation signals, termination signals, etc.

As used herein, the terms “complementary” and “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification and hybridization reactions, as well as detection methods that depend upon binding between nucleic acids.

Equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Q-replicase, MDV-1 RNA is the specific template for the replicase (Kacian et al., Proc. Natl. Acad. Sci. USA, 69:3038 [1972]; herein incorporated by reference). Similarly, in the case of T7 RNA polymerase, this amplification enzyme has a stringent specificity for its own promoters (Chamberlin et al., Nature, 228:227 [1970]; herein incorporated by reference). In the case of T4 DNA ligase, the enzyme will not ligate the two oligonucleotides or polynucleotides, where there is a mismatch between the oligonucleotide or polynucleotide substrate and the template at the ligation junction (Wu and Wallace, Genomics, 4:560 [1989]; herein incorporated by reference). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (Erlich (ed.), PCR Technology, Stockton Press [1989); herein incorporated by reference).

As used herein, the term “amplifiable nucleic acid” is used in reference to nucleic acids that may be amplified by any amplification method. It is contemplated that “amplifiable nucleic acid” will usually comprise “sample template.”

As used herein, the term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to a molecule (e.g., an oligonucleotide, whether occurring naturally as in a purified restriction digest or produced synthetically, recombinantly or by PCR amplification), that is capable of hybridizing to another molecule of interest (e.g., another oligonucleotide). When probes are oligonucleotides they may be single-stranded or double-stranded. Probes are useful in the detection, identification and isolation of particular targets (e.g., gene sequences). In some embodiments, it is contemplated that probes used in the present invention are labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular label. With respect to microarrays, the term probe is used to refer to any hybridizable material that is affixed to the microarray or provided with a chip for the purpose of detecting a “target” sequences in the analyte.

As used herein “probe element” and “probe site” refer to a plurality of probe molecules (e.g., identical probe molecules) affixed to a microarray substrate. Probe elements containing different characteristic molecules are typically arranged in a two-dimensional array, for example, by microfluidic spotting techniques or by patterned photolithographic synthesis, et cetera.

As used herein, the term “target,” when used in reference to hybridization assays, refers to the molecules (e.g., nucleic acid) to be detected. Thus, the “target” is sought to be sorted out from other molecules (e.g., nucleic acid sequences) or is to be identified as being present in a sample through its specific interaction (e.g., hybridization) with another agent (e.g., a probe oligonucleotide). A “segment” is defined as a region of nucleic acid within the target sequence.

As used herein, the term “oligonucleotides” or “oligos” refers to short sequences of nucleotides.

As used herein, the term “polymerase chain reaction” or “PCR” refers to the methods described in U.S. Pat. Nos. 4,683,195, 4,683,202, and 4,965,188, hereby incorporated by reference, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.” In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications. With PCR, it is possible to amplify a single copy of a specific target sequence in genomic DNA to a level detectable by the device and systems of the present invention.

As used herein, the terms “PCR product,” “PCR fragment,” and “amplification product” refer to the resultant mixture of compounds from at least two or more cycles o the PCR steps of denaturation, annealing and extension are complete. These terms encompass the case where there has been amplification of one or more segments of one or more target sequences.

As used herein, the terms “thermal cycler” or “thermalcycler” refer to a programmable thermal cycling machine, such as a device for performing PCR.

As used herein, the term “amplification reagents” refers to those reagents (such as, DNA polymerase, deoxyribonucleotide triphosphates, buffer, etc.), necessary for PCR-based DNA amplification.

As used herein, the terms “reverse-transcriptase” and “RT-PCR” refer to a type of PCR where the starting material is mRNA. The starting mRNA is enzymatically converted to complementary DNA or “cDNA” using a reverse transcriptase enzyme. The cDNA is then used as a “template” for a “PCR” reaction.

As used herein, the terms “restriction endonucleases” and “restriction enzymes” refer to bacterial enzymes, each of which cut double-stranded DNA at or near a specific nucleotide sequence.

As used herein, the term “recombinant DNA molecule” as used herein refers to a DNA molecule that is comprised of segments of DNA joined together by means of molecular biological techniques.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell genome in proximity to neighboring genes; RNA sequences, such as a specific mRNA sequence encoding a specific protein, are found in the cell as a mixture with numerous other mRNAs that encode a multitude of proteins. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

As used herein the term “coding region” when used in reference to a structural gene refers to the nucleotide sequences that encode the amino acids found in the nascent polypeptide as a result of translation of a mRNA molecule. The coding region is bounded, in eukaryotes, on the 5′ side by the nucleotide triplet “ATG” that encodes the initiator methionine and on the 3′ side by one of the three triplets that specify stop codons (i.e., TA, TAG, TGA).

As used herein, the terms “purified” and “to purify” refer to the removal of contaminants from a sample.

The term “recombinant DNA molecule” as used herein refers to a DNA molecule that is comprised of segments of DNA joined together by means of molecular biological techniques.

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide.

The terms “recombinant protein” and “recombinant polypeptide” as used herein refer to a protein molecule that are expressed from a recombinant DNA molecule.

As used herein the term “biologically active polypeptide” refers to any polypeptide that maintains a desired biological activity.

As used herein the term “portion” when in reference to a protein (as in “a portion of a given protein”) refers to fragments of that protein. The fragments may range in size from four amino acid residues to the entire amino acid sequence minus one amino acid.

As used herein, the terms “microbe” and “microbial” refer to microorganisms. In particularly preferred embodiments, the microbes identified using the present invention are bacteria (i.e., eubacteria and archaea). However, it is not intended that the present invention be limited to bacteria, as other microorganisms are also encompassed within this definition, including fungi, viruses, and parasites (e.g., protozoans and helminths).

As used herein, the term “reference DNA” refers to DNA that is obtained from a known organism (i.e., a reference strain). In some embodiments of the invention, the reference DNA comprises random genome fragments. In particularly preferred embodiments, the genome fragments are of approximately 1 to 2 kb in size. Thus, in preferred embodiments, the reference DNA of the present invention comprises mixtures of genomes from multiple reference strains.

As used herein, the term “multiple reference strains” refers to the use of more than one reference strains in an analysis. In some embodiments, multiple reference strains within the same species are used, while in other embodiments, “multiple reference strains” refers to the use of multiple species within the same genus, and in still further embodiments, the term refers to the use of multiple species within different genera.

As used herein, the terms “test DNA” and “sample DNA” refer to the DNA to be analyzed using the method of the present invention. In preferred embodiments, this test DNA is tested in the competitive hybridization methods of the present invention, in which reference DNA(s) from multiple reference strains is/are used.

The terms “sample” and “specimen” in the present specification and claims are used in their broadest sense. On the one hand, they are meant to include a specimen or culture. On the other hand, they are meant to include both a biological sample and an environmental sample. These terms encompasses all types of samples obtained from humans and other animals, including but not limited to, body fluids such as urine, blood, fecal matter, cerebrospinal fluid (CSF), semen, and saliva, as well as solid tissue. These terms also refers to swabs and other sampling devices that are commonly used to obtain samples for culture of microorganisms. Biological samples may be animal, including human, fluid or tissue, food products and ingredients such as dairy items, vegetables, meat and meat by-products, and waste. Environmental samples include environmental material such as water, (for example, fresh water, salt water, tap water, and the like), surface matter, soil, and industrial samples, as well as samples obtained from food and dairy processing instruments, apparatus, equipment, disposable, and non-disposable items. These examples are not to be construed as limiting the sample types applicable to the present invention.

As used herein, “conventional QPCR” and “QPCR” refer to “quantitative PCR,” that for the purposes of the present invention is a real-time PCR analysis, such as real-time PCR reactions that are performed by a Taqman® thermal cycling device and reaction assays by Applied Biosystems.

As used herein, “conventional PCR” and “PCR” refer to a nonquantitative PCR reaction, such as those reactions that take place in a stand-alone PCR machine without a real-time fluorescent readout.

As used herein, “isothermal amplification” refers to an amplification step that proceeds at one temperature and does not require a thermocycling apparatus.

As used herein, “Transcription-mediated amplification” and “TMA” refer to an isothermal nucleic acid amplification system for isothermic amplification of RNA using RNA polymerase.

As used herein, “Strand Displacement Assay” and “SDA” refer to an isothermal nucleic acid amplification system where cDNA product is synthesized from an RNA target.

As used herein, “Q-beta replicase” refers to an isothermal nucleic acid amplification system that uses the enzyme Q-beta replicase to replicate an RNA probe.

As used herein, “NASBA” refers to an isothermal nucleic acid amplification procedure comprising target-specific primers and probes, and the coordinated activity of THREE enzymes: AMV reverse transcriptase, RNase H and T7 RNA polymerase, for example, NASBA allows direct detection of viral RNA by nucleic acid amplification.

As used herein, “MicroElectroMechanical Systems” and “MEMS” refer to micrometer sized mechanical devices built onto semiconductor chips, such as pressure, temperature, chemical and vibration sensors, light reflectors and switches including optical switches that reflect light beams to the appropriate output port, as in a MEMS mirror.

As used herein, “Peltier cooling” and “Peltier unit” and “TEC” or “thermoelectric cooler” refer to active heat pumps, such that any of these devices are capable of cooling components below ambient temperatures. In one embodiment, a heat pump comprises stacked units of dozens up to hundreds of thermocouples laid out next to each other, allowing for a substantial amount of heat transfer away from a component of higher temperature.

As used herein, “integrated heater” refers to a small electronic heater comprising semiconductor material.

As used herein, “semiconductor” refers to a material that is neither a good conductor of electricity (such as copper) nor a good insulator (such as rubber) used in providing miniaturized components for taking up less space, faster and requiring less energy than larger components. Examples of common semiconductor materials are silicon and germanium and the like.

As used herein, “light-emitting diode” or “LED” refers to a semiconductor device that when electrically stimulated in the forward direction emits a form of electroluminescence as incoherent narrow-spectrum light.

As used herein, “organic light-emitting diode” or “OLED” refers to a light-emitting diode (LED) in which the emissive layer comprises a thin-film of organic compounds.

As used herein, “OEL” or “organic electro-luminescence” refers to a type of light-emitting diode (LED) in which the emissive layer comprises a thin-film of organic compounds.

As used herein, “Luminance” or “spectral luminance” refers to observed brightness measured in footlambert units of cd/m2 or cd/ft2, 1 of these units may also be referred to as a “nit.”

As used herein, “footlambert” or “fL” or “fl” refers to a unit of measurement of luminance in U.S. customary units where 1 footlambert equals π⁻¹ candela per square foot, or 3.4262591 candela per square meter (nits or cd m²), for example, 1 footlambert=3.43 candela meter² (cd m²).

As used herein, “candela” or “cd” refers to a base unit of luminous intensity such that power emitted by a light source in a particular direction, with wavelengths weighted by the luminosity function, provides a standardized model of the sensitivity of the human eye.

As used herein, “pound” or “lb” or “avoirdupois pound” refers to a unit of mass (or weight) equal to 16 ounces or 16 avoirdupois ounces that is equal to approximately 453.59 grams.

As used herein, “peripheral” refers to a device, such as a computer device, for example, a CD-ROM drive or wireless communication chip, that is not part of the essential computer, i.e., the memory and microprocessor. Peripheral devices can be external, such as a mouse, keyboard, printer, monitor, external Zip drive or scanner or internal, such as a CD-ROM drive, CD-R drive or internal modem. Internal peripheral devices may be referred to as “integrated peripherals.”

As used herein, “light source” in reference to an illuminating (illumination) light source refers to an excitation light source for exciting electrons in a fluorescent molecule.

As used herein, “chamber” or “holder” in reference to a sample, such as a biological sample chamber, refers to an area capable of comprising a biological sample, such as a special area, actual holder, and the like.

As used herein, “transparent” in reference to optical, refers to the capability of allowing light to pass through a substance of matter, such that optically transparent for use in the present inventions is at least 80%, 90%, 95%, and up to 100% optically transparent to light generated by compositions and methods of the present inventions.

As used herein, “detecting” in reference to light emitted a fluorescent compound refers to the capability of sensing an optical signal emitted from the fluorescent compound.

### GENERAL DESCRIPTION OF THE INVENTION

The present invention provides compositions providing and methods using a fluorescence detection device, comprising an electroluminescent light (EL) source, for measuring fluorescence in biological samples. In particularly preferred embodiments, the present invention provides an economical, battery powered and hand-held device for detecting fluorescent light emitted from reporter molecules incorporated into DNA, RNA, proteins or other biological samples, such as a fluorescence emitting biological sample on a microarray chip. Further, a real-time hand-held PCR Analyzer device comprising an EL light source for measuring fluorescence emissions from amplified DNA is provided.

The present invention provides compositions and methods for fluorescence detection devices for measuring fluorescence emitted by biological samples. In preferred embodiments, the present invention provides a commercially economical fluorescence detection device comprising an electroluminescent light source for detecting fluorescent light emitted from reporter molecules incorporated into DNA, RNA, proteins or other biological samples. In additional embodiments, the fluorescence detection device is battery powered and portable. In one embodiment, the invention provides a hand-held device for fluorescence detection of a biological sample, such as a PCR chip. In particularly preferred embodiments, the present invention provides a commercially economical hand-held device for fluorescence detection of real-time PCR amplification reactions. In particularly preferred embodiments, the present invention provides a fluorescence detection device capable of PCR based amplification reactions, comprising an electroluminescent light source, an integrated heater and a Peltier cooling unit. The inventors further contemplate the use of EL film based detection units for using microarray chips comprising primers and probes for identifying pathogens, in particular water pathogens, Hashsham et al., Microbe Volume 2, Number 11, 2007, herein incorporated in its entirety.

Portable diagnostic tools for fluorescence based microbial detection of genetic and functional signatures are essential for fast point-of-use clinical and environmental applications. Currently, several companies offer hand-held and/or portable diagnostic devices for testing microbial populations specifically in water, but detect limited types of bacteria. One example, for detecting total Coliform and E. coli (Hach Co.), is a bulky Manchester Environmental Laboratory (MEL)/most probable number (MPN) Method Laboratory Kit. This kit includes a portable incubator, portable UV lamp, and consumables for 50 tests, media is not included, that provides a qualitative test that indicates only the presence or absence of a coliform, including an E. coli subset, in 24 to 48 hours. Another example, with a reported shorter 30 minute read-out on chosen microorganisms, such as anthrax bacteria, is a GeneXpert® System (Cepheid) for providing real-time polymerase chain reaction (PCR) to amplify and detect target DNA from unprocessed environmental samples. This system includes a processing unit that is 11.5″ wide×14″ high×12.25″ deep as described in “GeneXpert: The world's only fully integrated real-time PCR system” (Cepheid Technical publication 0112-02, herein incorporated by reference). This system comprises a SmartCycler® type device that provides real-time PCR reactions for identifying DNA/RNA from prepared biological samples. A SmartCycler® (Cepheid) is 12″W×12″L×10″H and weighs at least 22 lbs.

Thus, significant reductions in diagnostic device cost and per sample cost, in addition to reducing analysis time and increase in target identification are needed for the economical use of hand-held or portable diagnostic fluorescence based detection devices. Critical parameters for the development of such detection devices include lowering weight, type of fluorescent excitation and imaging technology, lowering cost, lowering size, lowering power consumption while increasing safety, such as eliminating the use of UV light, and increasing sensitivity, such as increasing the number of different types of detectable microorganisms and providing genetic and functional signatures of these microorganisms.

A critical parameter affecting size, weight, and economic constraints for providing an economical fluorescence based Hand-held or portable diagnostic device is the light source used for sample illumination, in particular for fluorescence-based excitation. One solution for providing a small, lightweight and economical light source is to use a LED-based illumination device.

Thus several companies have provided LED-based devices as light sources for illuminating samples comprising fluorescent dyes. For one example, a portable microprocessor-based LED water analyze is CHEMetrics's V-2000 Multi-analyte Photometer or SAM—Single Analyte Photometer Kit using CHEMetrics' Vacu-vials® self-filling ampoules. However these devices and kits primarily test for identifying analytes related to bacteria contamination not the actual identification of bacteria or microbes.

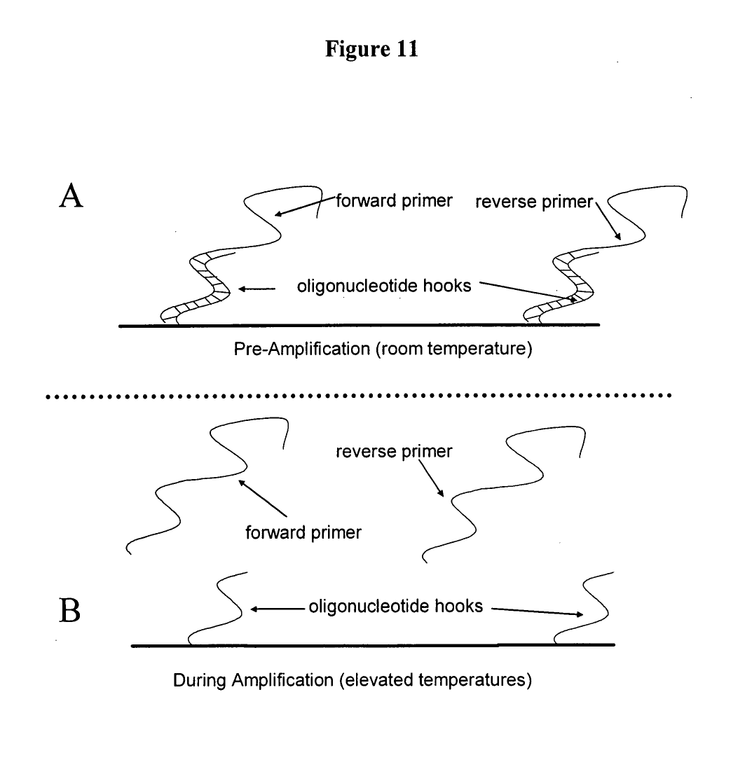

Further, several companies offer hand-held and/or portable diagnostic devices and kits for using molecular techniques incorporating florescent molecules/dyes for identifying types of bacteria in environmental samples. For the latter purpose, there are at least five PCR machines comprising fluorescent detection devices commercially available: Bio-Seeq™'s HANAA (Smiths Detection), RAPID® and RAZOR™ (Idaho Technology Inc.) and Smartcycler™ and GeneExpert™ System (Cepheid Inc.). Of these, three are advertised as hand-held and/or portable devices; Bio-Seeg™s HANAA (Smiths Detection), RAPID® and RAZOR™ (Idaho Technology Inc.). However these five machines are heavy, at least 6.5 pounds in weight, large, at least 17×11×23 cm (h×d×w), with a restricted range of sample numbers, limited target identification and little information for providing a genetic and functional signatures, such as information on the presence of multiple types of bacteria, the presence of multiple bacterial species within a genus or whether bacteria are in a log growth phase or static. See, FIGS. 11-16 for further sample based and cost comparisons.

In particular, these commercial products and the devices of the present inventions are designed to provide conventional or real-time PCR assays, such as qPCR (quantitative PCR), for detecting biological pathogens that are designed to be performed outside of BSL 3 (Biosafety Level 3) containment (as described in Biosafety in Microbiological and Biomedical Laboratories (BMBL) 4th Edition ed, Richmond and McKinney published by the U.S. Department of Health and Human Services Centers for Disease Control and Prevention and National Institutes of Health Fourth Edition, May 1999 US Government Printing Office Washington: 1999) either in a laboratory or on portable devices taken to the site of the problem.

One example of a conventional PCR analyzer is a Bio-Seeg™ (Smiths Detection Handheld PCR Instrument) Handheld Advanced Nucleic Acid Analyzer (HANAA) uses two light emitting diodes (LED) to provide greater than 1 mW of electrical power at wavelengths of 490 nm (blue) and 525 nm (green). HANAA is a portable real time thermal cycler unit that weighs less than 1 kg (about 6½ pounds and the approximate size of a book) is 28×9×18 cm (11×3.5×7 inches) that uses silicon and platinum-based thermalcycler units to conduct rapid heating and cooling of plastic reaction tubes. Results are displayed in real time as bar graphs, and up to three, 4-sample assays can be run on the charge of the 12 V portable battery pack. HANAA is powered by batteries, vehicle adapter, or AC plug and can test up to six different samples simultaneously (See, review, Higgins et al., (2003) Biosensors and Bioelectronics, 18(9):1115-1123; Lawrence Livermore National Laboratories. “Chemical and Biological Detection Technologies.” (15 Jan. 2003); Ronald Koopman et al. HANAA: Putting DNA Identification in the Hands of First Responder; all of which are herein incorporated by reference.).

Another example of an LED illuminated real-time PCR Analyzer is a Ruggedized Advanced Pathogen Identification Device (R.A.P.I.D.®) PCR machine (Idaho Technology). R.A.P.I.D.® is a portable device of 50 pounds and requiring a 110-volt power source to identify biological agents in under 30 minutes.

A related device is a stand-alone, battery-operated real-time PCR thermal cycler with built in analysis and detection software RAZOR™, comprising a fan cooled thermal cycler (http://www.idahotech.com/RAZORTm/features.html), that is 8 pounds in weight, 6.6×4.4×9.1 inch/17×11×23 cm (h×d×w) and reported to analyze 12 samples in 22 minutes running only on battery power.

A solution contemplated by the inventors for providing a small, lightweight, economical and safe light source is using electroluminescent film (ELF) based illumination fluorescent detection devices as described herein. EL emitted light is in the visible spectrum and can be directly viewed without damaging human eyes.

One commercially available bench-top device for detecting EL type illumination is a BioVeris M-SERIES MIM Analyzer (BioVeris Corporation). However, this device measures EL illumination produced by an EL antibody tagged target unlike the devices of the present invention wherein the EL material is a device component providing a light source for fluorescent illumination.

With the appropriate combination of EL and excitation/emission light filters, light emitted from electroluminescent film (ELF) satisfies the critical parameters of a portable illumination device. In one embodiment, blue light emitted by an ELF lamp excites a number of fluorophores/dyes including SYBR Green, SYBR gold, SYBR safe, EvaGreen, Green fluorescent proteins, Fluorescein, and the like.

The inventors further contemplated versatility of ELF (such as thickness and size, 0.2 mm×any desired spatial dimension; zero heat generation; long life of over 10,000 hours of light emission; and low cost) are ideal for use in portable diagnostic devices and inexpensive sample analysis devices in the laboratory and for use under field conditions, including as diagnostic devices for detecting biological warfare agents. Results shown herein, demonstrate that illuminated ELF, as in an ELF lamp, provides highly sensitive fluorescence that can be documented with a CCD camera or photographed as a demonstration of the image observed with a naked human eye.

Including rechargeable batteries and a DC to AC inverter, the inventors contemplate a luminescent device comprising elements that cost less than a total of $25 U.S. and further these elements will be customized based on a desired spatial viewing area. Wherein said low cost is the cost for purchasing the detector elements.

A contemplated objective for the fluorescence detection device of the present inventions is to provide a Hand-held and/or portable fluorescence detection device of low cost.

A contemplated objective for the fluorescence detection device of the present inventions, is to provide a Hand-held and/or portable fluorescence detection device of less than 4301 sq. cm (264.26 sq. inch), more preferably less than 2000 sq. cm, more preferably less than 1000 sq. cm, more preferably less than less than 500 sq. cm, even more preferably less than 50 sq. cm, even more preferably less than 20 sq. cm.

A Hand-held and/or portable fluorescence detection device is up to 6.5 inches in diameter, preferably 5 inches, x a thickness of 4.3 inches, preferably 3 inches. In one embodiment, the device additional comprises up to a 4-inch handle.

A contemplated objective for the fluorescent detection device of the present inventions is to provide a Hand-held and/or portable fluorescence detection device of low weight, less than 6.5 lbs (104 oz. and 2.95 kg), not including an external power source. Accordingly the weight is more preferably less than 3 lbs (48 oz. and 1.36 kg), more preferably less than 2 lbs (32 oz. and 907 g), more preferably less than 1 lb (16 oz. and 454 kg), and even more preferably less than 0.5 pound (8 oz. and 227 g).

In one embodiment, the inventors contemplate a Hand-held device of the present invention the size and weight of a Blackberry® 7250 at 4.90 oz and 11.8 sq. inches. In one embodiment, the inventors contemplate a Hand-held fluorescence device of the present invention the size and weight of a Palm® Treo™ 700 p at 6.4 ounces (180 g) and 10.3 sq. inches.

A contemplated objective for the fluorescence detection device of the present inventions, is to provide a Hand-held and/or portable PCR Pathogen Analyzer device of low cost.

In one embodiment, the inventors contemplate a Hand-held fluorescence device of the present invention the size and weight of a Blackberry 7250 at 4.90 oz and 11.8 sq. inches. A contemplated objective for the fluorescence detection device of the present inventions, is to provide a Hand-held and/or portable PCR Pathogen Analyzer device of less than 4536 cm² (269.5 in²).

Accordingly, a PCR Pathogen Analyzer device of the present invention is more preferably less than 2000 cm², more preferably less than 1000 cm², more preferably less than less than 500 c cm², more preferably less than 269.5 cm² (264.26 in²), 50 sq. cm (19.685 sq. inches), even more preferably less than 20 sq. cm (7.874 sq. inches). In one embodiment, the inventors contemplate a Hand-held device of the present invention the size and weight of a Blackberry® 7250 at 4.90 oz and 11.8 sq. inches. The PCR Pathogen Analyzer device is up to 6.5 inches in diameter, preferably 5 inches, x a thickness of 4.3 inches, preferably 3 inches. In one embodiment, the device additional comprises up to a 4-inch handle.

A contemplated objective for the fluorescence detection device of the present inventions is to provide a Hand-held and/or portable fluorescence detection device of low weight, less than 6.5 lbs (104 oz. and 2.95 kg), not including an external power source. Accordingly the weight is more preferably less than 3 lbs (48 oz. and 1.36 kg), more preferably less than 2 lbs (32 oz. and 907 g), more preferably less than 1 lb (16 oz. and 454 kg), and even more preferably less than 0.5 pound (8 oz. and 227 g. In one embodiment, the inventors contemplate a Hand-held device of the present invention the size and weight of a Palm® Treo™ 700 p at 6.4 ounces (180 g) and 10.3 sq. inches.

Thus a fluorescent detection device or PCR Pathogen Analyzer device of the present inventions that use electroluminescent (EL) film based fluorescent detection is estimated to be over 10× less costly and 450× thinner than conventional devices such as transilluminators and UV stations.

The inventors contemplate that EL film based fluorescent detection devices of the present invention would provide safe and economical Bench-Top fluorescent imaging devices. In one embodiment, a Bench-Top fluorescent imaging device of the present invention would replace conventional transilluminators and UV stations.

The inventors contemplate Hand-held and/or portable EL film based fluorescent detection devices of the present invention. Thus in another embodiment, EL film based fluorescent detection devices of the present invention would provide Hand-held and/or portable fluorescent detectors. Additionally, the inventors contemplate providing a real-time PCR pathogen analyzer of the present invention comprising an EL based illumination source for providing real-time PCR analysis. The inventors further contemplate that the EL film based real-time PCR pathogen analyzer of the present invention would replace portable PCR based devices and other types of detection devices currently used for biological detection in environmental and other types of samples.

Specifically the inventors contemplated that unlike the currently available PCR based pathogen analyzers, an EL film based real-time PCR pathogen analyzer of the present invention would be safer, more cost-effective and provide more information per sample. See, FIGS. 11-16.

### DETAILED DESCRIPTION OF THE INVENTION

The inventors believe that combining microfabrication techniques, such as semi-conductor and nanotechnology, with biochemical procedures will result in highly sensitive and specific methods for detecting pathogenic microorganisms. In particular, the inventors contemplate identifying pathogenic microorganisms in water samples.

In order to achieve these goals, the inventors contemplate providing EL-based diagnostic fluorescent detection devices for providing assays and results with one or more of the following characteristics: the assays will be performed by persons of either experienced personal or limited training (for example, soldiers, field technicians, and the like). Further that such assays will be performed using quality-controlled standardized reagents and protocols that are internationally consistent with results that should be obtained in an hour or less; assays may be relayed in real-time or delayed time for review on a desk-top computer or over the Internet.

The following is a detailed description of EL-based Bench-top and EL-based Hand-held fluorescent detection devices of the present invention, including non-limiting examples of device elements, in the following sections: I. EL-based Light Sources, II. Bench-top and Hand-held EL-based Florescence Detection Systems, III. EL-based Real-time PCR Analyzers, IV. Methods relating to use of EL-Based Detectors and Analyzers and V. Economic Feasibility.

### I. Electroluminescent (EL)—Based Light Sources.

The present invention is directed to the use of an economical and human safe light source for providing florescent detection devices. In one embodiment, the light source is an electroluminescent light (EL) source that may be referred to as an electroluminescent (EL) lamp. In one embodiment, the EL light source is an AC thin-film electroluminescent light source. In one embodiment, the light source is electroluminescent (EL) film (ELF). In a preferred embodiment, the light source is a commercially available electroluminescent film. Many types of ELF are available comprising flexible films, such as polyethylene terephthalate (PET) film.

A. Electroluminescent (EL) Light Source.

Electrical current or exposure to an electrical field will induce the emission of electroluminescence from an EL source, such as an EL film (ELF) in the form of visible light i.e. ON, wherein light output is dependent upon voltage and frequency producing an ELF lamp.

The inventors contemplated using an electroluminescent light (EL) source, in particular EL film, for the fluorescent detection devices of the present inventions. In one embodiment, EL material, such as a dielectric substance and a phosphor, are enclosed between two electrodes. In one embodiment, at least one electrode is transparent to allow the escape of the produced light. In one embodiment, the transparent electrode is glass coated with indium oxide or tin oxide. In one embodiment, the nontransparent or back electrode is or is coated with reflective metal. In one embodiment, the front and back electrode is transparent to allow the escape of the produced light.

The following characteristics of ELF contribute to the detection systems of present inventions; ELF does not catastrophically or abruptly fail unlike filament or fluorescent lighting; consumes 75-90% less power than other point light sources, such as a UV point light source; operates at a low temperature with little or no heat generation, unlike conventional LED lights; is safe for direct viewing by human eye; waterproof; uses no hazardous materials; long service life, as in over 10,000 hours; is maintenance free, etc. In particular, ELF is thin and flexible, generates light without heat, can be dimmed, does not include a filament, is light weight, for example, one type of ELF weighs 4 ounces per square foot.

The EL based light source may be any shape. Preferably, the light source is made of flexible material that may be cut into a desired size or shape without damage to the light source. The preferred shape is square, however, a light source of any other shape can be employed. For example, a preferable shape of the light source allows for optimal excitation of the biological sample in the detection devices of the present inventions.

In one embodiment, ELF is cut to fit the portable device, for example, the film is cut with a knife, plotter, LASER and the like.

1. EL Light Sources.

An EL source may be a film or a sheet of film, both referred to as “ELF.” Characteristics of ELF that contribute to the present inventions include but are not limited to thickness, as in the ability to form thin layers, for an example, 0.25 mm-0.5 mm thick.

ELF is on sale as sheets, panels, strips that can be cut to any size or shape. ELF may also be bent to configure to a desired shape or design. ELF is lightweight, for example, one type of EFL weighs 2 oz/sq-ft. (KNEMA, LLC, Luminous Film), see, Table 1 for further examples.

2. Additional Types of EL Light Sources.

The inventors do not intent to limit the types of EL sources used in the present inventions. In some embodiments, the light source is an organic light-emitting diodes (OLEDs) Yang (2005) Colloids and Surfaces A: Physicochemical and Engineering Aspects 257-258:63-66.

The inventors' further contemplate the use of a variety of electroluminescent light sources, including but not limited to those described herein, and electroluminescent light based upon two-photon single-photon and single-molecule optoelectronics, see, Lee et al., (2005) Acc Chem Res. 38(7):534-41; Gonzalez et al., (2004) Phys Rev Lett. 93(14):147402; (2004) Phys Rev Lett. 93(15):159901; Lee et al., (2002) Proc Natl Acad Sci U.S.A. 99(16):10272-5. Epub 2002 Jul. 29; Gonzalez et al., Phys Rev Lett. (2004) 93(14):147402, Epub 2004 Sep. 27, Erratum in: (2004) Phys Rev Lett. 93(15):159901; and Lee et al., (2002) Proc Natl Acad Sci U.S.A. 99(16):10272-5, Epub 2002 Jul. 29; all of which are herein incorporated by reference.

B. Thin Film EL (TFEL) Lamp.

Initially, EL lamps were made on at least 7 mil (0.19 mm) thick substrates, such as PET, however thinner lamps are produced, such as for consumer devices. Thus the inventors contemplate using thin-film EL light sources, wherein said thin-film refers to a layer of colloidal substance (such as one or more of a phosphor, or dielectric substance) equal to 0.19 mm or less, as deposited upon an ITO coated surface. Even further, nanostructured thin films are contemplated for use in the present inventions, such as NS—ZnS:Mn, ZnS:Mn/Si3N4 multilayers with thicknesses of 1.9-3.5 nm described in Toyama, et al., (2000) Mat. Res. Soc. Symp. Proc. Vol 621:Q4.4.1; and further examples, Ohmi, et al., (1998) Applied Physics Letters, 73(13):1889-1891; and Minami, et al., (2001) Journal of Vacuum Science & Technology A: Vacuum, Surfaces, and Films 19(4):1742-1746; all of which are herein incorporated by reference.

Further, thin film EL lamps comprising high-voltage silicon switches in integrated circuit (IC) form have led to improved efficiencies. In addition, the improved intrinsic efficiency of thin film lamps and phosphors has allowed a new generation of inexpensive and compact IC-based, relatively noise-free EL lamp drivers to be developed.

C. Electroluminescent Film Inverter Drivers.

In general, Electroluminescent (EL) Film provides even illumination while consuming relatively little electric power, such as electrical power supplied by in-line electrical current, such as wall current, or batteries. A variety of electrical sources may be used to power at least the ELF portion of the EL devices of the present inventions. EL Film and further EL Film-based devices may be powered by AC or DC.

1. In-Line Electrical Current.

In one embodiment, EL Film is powered by electrical connections to commercial power sources or generators. In one embodiment, EL film is in electrical combination with an AC adapter/inverter/driver capable of being plugged into a standard 120V/60 Hz outlet. For example, an EL driver is a 12V DC Wall Transformer, External Inverter, 500 mamps, ($9.25 U.S.) or a 12V DC External Inverter Wall transformer 1.2 amp, ($21.75 U.S.), or EL Display Drivers such as those produced by Zywyn Corporation.

Wherein the AC current is transformed to 12V DC current and goes into the inverter driver, in which the DC current is “inverted” back into AC in order to provide higher voltage or frequency, such as 120V or 400-1600 Hz. The voltage and frequency required from the inverter will depend on the size of the EL sheet. In one embodiment, an EL is in electrical combination with a standard 12V AC adapter. Light output and color are functions of the voltage and frequency applied, respectively. Therefore, a higher frequency is used to provide a greater output of blue hue. To reduce power consumption and life expectancy, the frequency and voltage should be minimized while sustaining an optimal light output for detecting PCR amplification. An optimal voltage range of 100 to 240 VAC and an approximate frequency of 645 Hz is recommended by many manufacturers for drawing 0.0003 amps per square inch of illuminated surface.

2. Battery Driven EL Device.

The inventors contemplate a portable device free from the constraints of commercial power sources or generators. Thus EL light sources, inverters, ELF drivers, and the devices described herein are driven by battery operated units. Examples include, an ELF driver, such as a Continuous Double Core driver (AS&C CooLight), and Electroluminescent Inverter Drivers for 3V—AA inverter, 6V, 9V and 12V and 110VAC applications (Being Seen Technologies, Being Seen.com). In one embodiment, an EL is in electrical combination with a 3V or 9V or 12V battery cell, such as an alkaline battery. In one embodiment, an EL is in electrical combination with a car battery.

### II. EL-Based Bench-Top and Hand-Held Florescence Detection Systems.

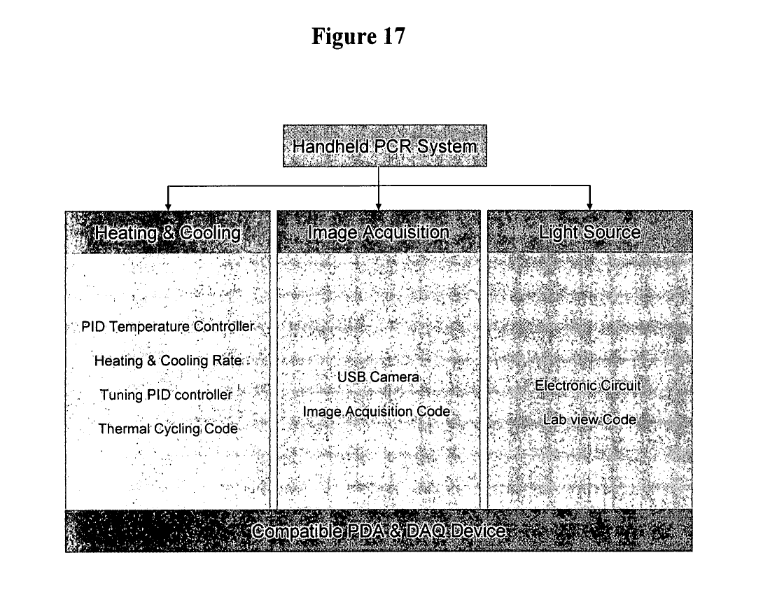

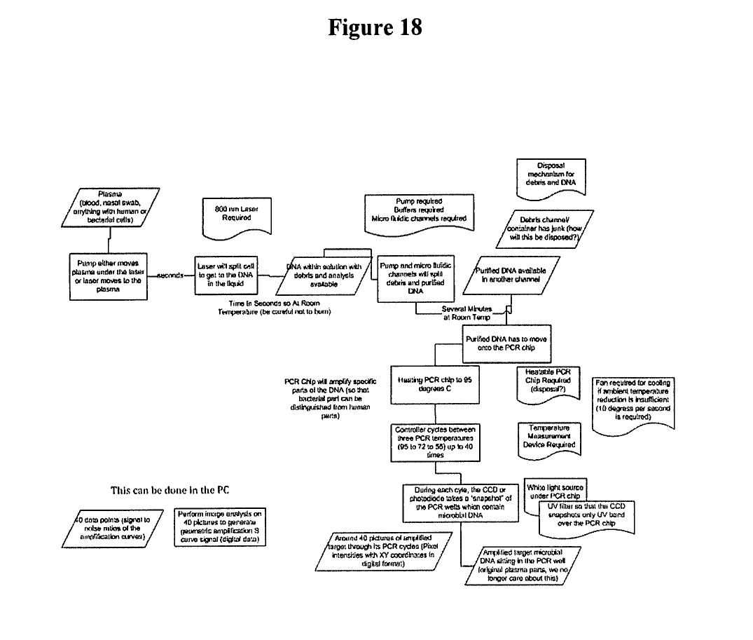

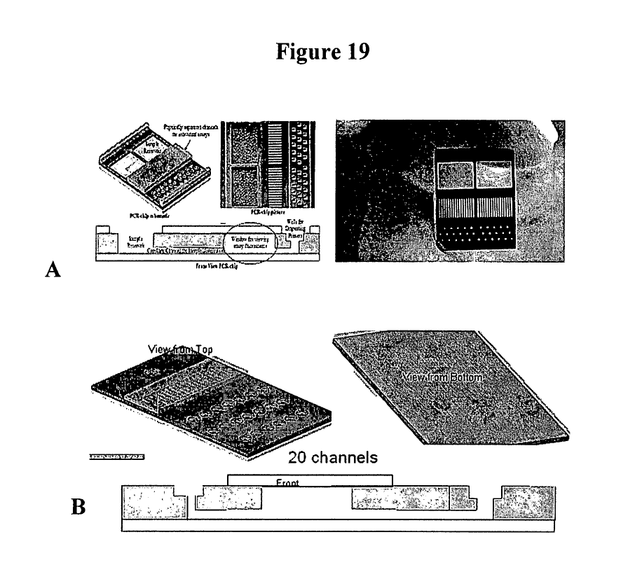

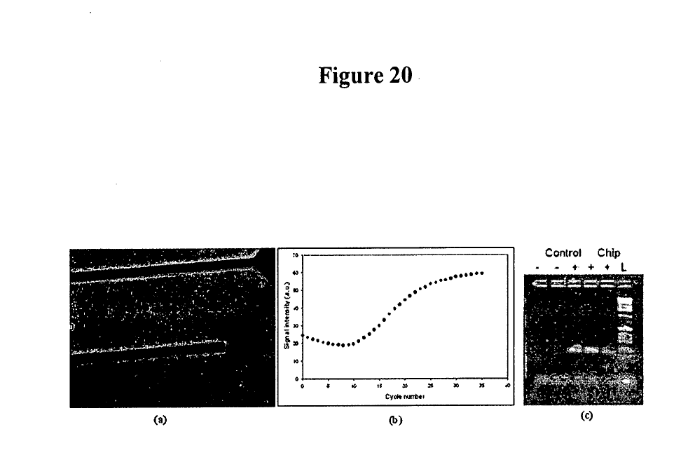

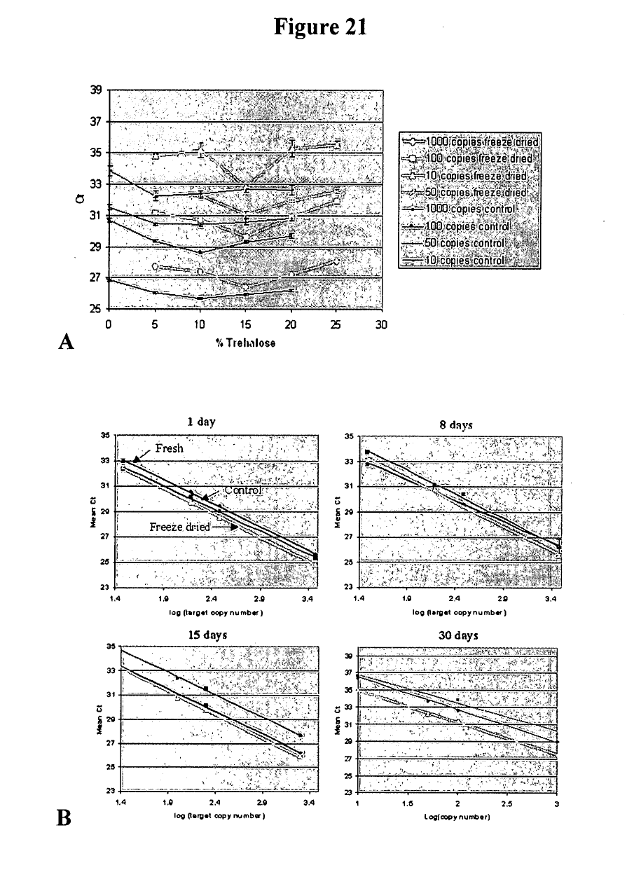

The following overview shows exemplary descriptions and components and are not intended as limiting examples (FIGS. 17 and 18, wherein Rectangles depict an activity, polygon depicts materials, and boxes with curved side depict contemplated electronic and microfluidic components).

A. EL-Based Bench-Top Florescence Detection Systems

The present invention is different from commercially available devices using

EL based light sources. Commercially available devices using EL based light sources are expensive stationary duel detection devices that additionally emit potentially hazardous UV light such as UV transilluminators and UV stations for detecting fluorescent emissions. In one example, a duel EL and UV based light source device is a “FOTO/PRO 1000 White Light Transilluminator” or “FOTO/UV® 450 Ultraviolet Transilluminator” uses both an EL excitation source and a 488 nm argon-ion laser excitation source for imaging protein gels, autorads, and microtiter plates, for viewing up to 26×38 cm surfaces or TLC analysis, viewing DNA agarose gels stained with ethidium bromide or SYBR® Green I nucleic acid gel stain, “UV shadowing” for visualizing nucleic acids on gels, respectively. Fluorescence detection is recorded by spectrograph and CCD camera. In another example, an “Electroluminescent FOTO/Phoresis® White Light Transilluminator” is available for viewing Coomassie blue-stained protein mini gels, methylene blue-stained DNA gels and colorimetric reactions in microtiter plates, where using a photographic hood and a hand-held FCR-10 camera produces a 1:1 Polaroid photograph, and with FOTO/Analyst® CCD camera with hood and filter. No focusing is required. In seconds the Thermal Printer provides you with a continuous-tone black and white print (256 gray scale quality). A CCD video camera mounted in support frame and much more. UV blocking eyeglasses UV Blocking Cover EL illumination (see EL description below), allows the white light both UV and White Light.

The inventors provide a Bench-Top EL-based illumination system. Further, this bench-top system is inexpensive and easy to use as described in Examples 1 and 2 below.

B. EL-Based Hand-Held Florescence Detection System.

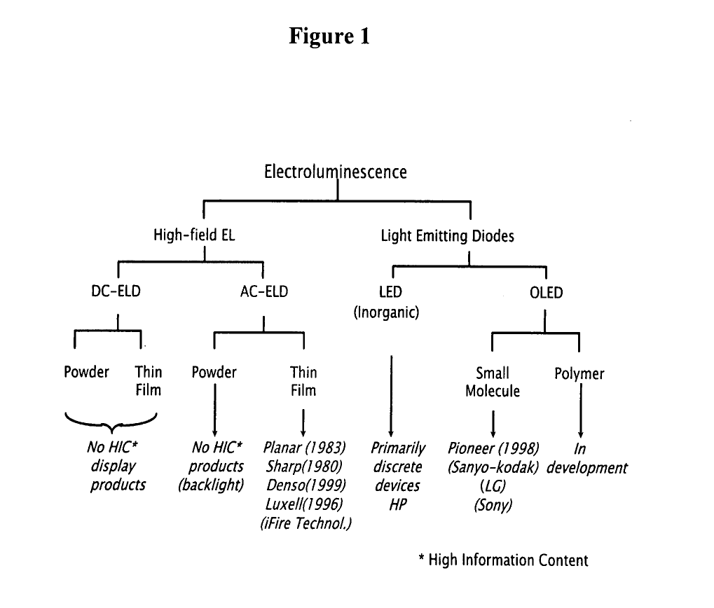

The inventors contemplate EL-based hand-held florescence detection devices of the present inventions. An EL film-based hand-held florescence detection device (ELFFD) is contemplated as a Hand-held and/or portable alternative to a bench-top fluorescent plate reader. In one embodiment, an ELFFD device is described below. In FIGS. 1 to 2 of the accompanying drawings there is schematically depicted a detection device 10. The device 10 of this embodiment is configured as a “hand-held.” The device 10 is in electrical combination with an external or internal inverter/power supply 15 or 16 in electrical combination with an electroluminescent assembly 22 that is in electrical combination with an internal processor 19, a CMOS battery, an optional RFID transponder, an external keypad 27, a USB port 14, RAM, internal memory and any additional internal components of the present inventions.

1. EL-Based Device.

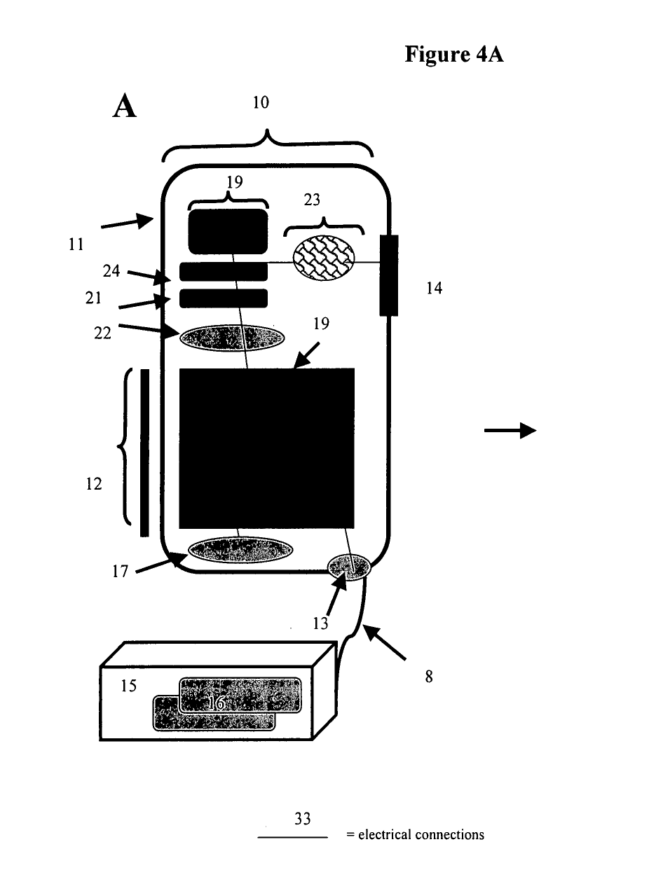

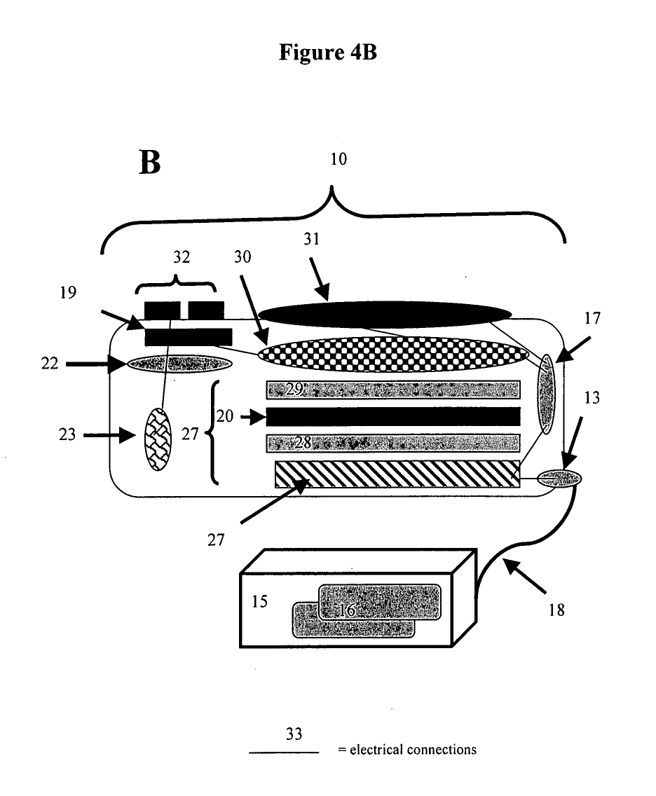

A basic description of an exemplary EL-based device of the present invention is provided in FIGS. 4, 17 and 18. The device 10 comprises a casing/body, such as an external case 11, and a sample slot 12 (e.g. for accommodating a PCR chip following PCR reaction). In some embodiments, access to the sample slot 12 may be located in other locations. For example, the sample slot may be accessed by raising the LCD display. The device further comprises, in electrical combination: port for battery cord 13, USB port 14, inverter/power supply 15, battery 16, internal battery 17 (optional), power cord 18, sample chamber 19 (e.g. PCR Chip or other biological sample), sample 20 (e.g. PCR chip or other biological sample), processor 21, RAM 22, internal memory 23, CMOS battery 24, wireless communication chip 25, electroluminescent assembly 26, electroluminescent emitter 27, excitation filter 28, emission filter 29, CMOS or CCD image detector 30, external visual display (LCD) 31, external key pad 32, and exemplary electrical connections 33.

TABLE XX
Key for schematics in FIG. 4A and 4B.

| No. | Component |
|---|---|
| A | Internal Front View |
| 10 | Detection Device |
| 11 | Casing/Body |
| 12 | Sample slot (e.g. PCR chip following PCR reaction, for inserting |
| | a PCR chip) |
| 13 | Port for battery cord |
| 14 | USB port |
| 15 | Inverter/power supply |
| 16 | Battery |
| 17 | Internal Battery (optional) |
| 18 | Power cord |
| 19 | Sample chamber (e g PCR Chip or other biological sample) |
| 20 | Sample (e g PCR Chip or other biological sample) |
| 21 | Processor |
| 22 | RAM |
| 23 | Internal Memory |
| 24 | CMOS Battery |
| 25 | Wireless communication chip (optional) |
| B | Internal Side View |
| 26 | Electroluminescent (EL LAMP) assembly |
| 27 | Electroluminescent emitter |
| 28 | Excitation Filter |
| 29 | Emission Filter |
| 30 | CMOS or CCD image detector |
| 31 | External visual display (LCD) |
| 32 | Key pad |
| 33 | Electrical connections |

2. Electroluminescent Assembly.

An exemplary electroluminescent assembly 22 comprises an electroluminescent emitter (capacitor) 23, in optical combination with excitation filter 23, sampling chamber 18, emission filter 25, CMOS or CCD image detector 26 and is in electrical combination with external visual display 27.

C. Data Capture and Analysis.

In addition, analyzers of the present inventions would provide real-time read-out displays and analysis of results. The digital data stream obtained by the detector would be processed by a microcontroller. The inventors contemplate programming the microcontroller for providing a visual and digital output for each well or assay. The visual output is sent to an LCD display. For example, a visual output comprising one positive well or assay, is shown below:

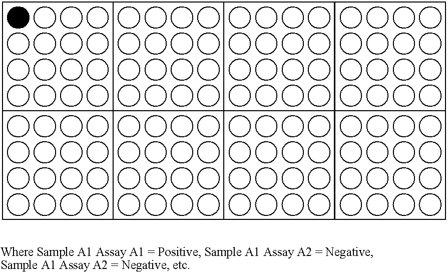

For providing immediate results, such as for testing for the presence of E. coli O157:H7 or anthrax bacterium or spores, the visual output is sent to an LCD display shows the name of the organism with a positive/negative or present/absent answer.

D. Analysis Software.

The inventors contemplate fluorescent detection devices of the present inventions further comprising software for providing conventional and/or real-time qPCR analysis and read-outs. In one embodiment, such software would provide a positive/negative or present/absent answer. In one embodiment, such software would provide a qualitative answer. Software contemplated for use in the present invention provides sample analysis capabilities at the level of currently available PCR analysis software or greater capabilities for analysis. For example, software of the present invention is contemplated to provide a clear analysis between background fluorescent level and a positive fluorescent signal. In one example, a device of the present invention uses software that provides such functions are present in Affymetrix GeneChip® Operating Software (GCOS), wherein GCOS automates the control of GeneChip® Fluidics Stations and Scanners. In addition, GCOS acquires data, manages sample and experimental information, and performs gene expression data analysis. GCOS supports the GeneChip® DNA Analysis Software (GDAS), GeneChip® Genotyping Analysis Software (GTYPE), and GeneChip® Sequence Analysis Software (GSEQ) for resequencing and genotyping data analysis. In one embodiment, a fluorescent device of the present invention comprises GCOS, GDAS, GTYPE, GSEQ, and the like. The inventors contemplate a variety of data read-outs, including but not limited to the LED display of the devices of the present inventions. The inventors further contemplate transferring images to a separate computer using one or more of a USB cable, a memory card or wireless communication devices.

### III. EL-Based PCR Analyzer.

The EL-based real-time PCR analyzer devices of the present invention are contemplated by the inventors to provide an inexpensive, fast and accurate handheld device for conventional or on-chip DNA amplification and detection based on PCR reactions. In one embodiment, the inventors contemplate an EL-based hand-held conventional PCR device, for example, to amplify DNA as in conventional PCR, RT-PCR, and the like. In another embodiment, the inventors contemplate an EL-based real-time hand-held PCR device, such as a quantitative PCR device. In yet a further embodiment, the inventors contemplate an EL-based real-time Hand-held isothermal PCR device, for example, isothermal amplification of DNA, isothermal RT-PCR, and the like.

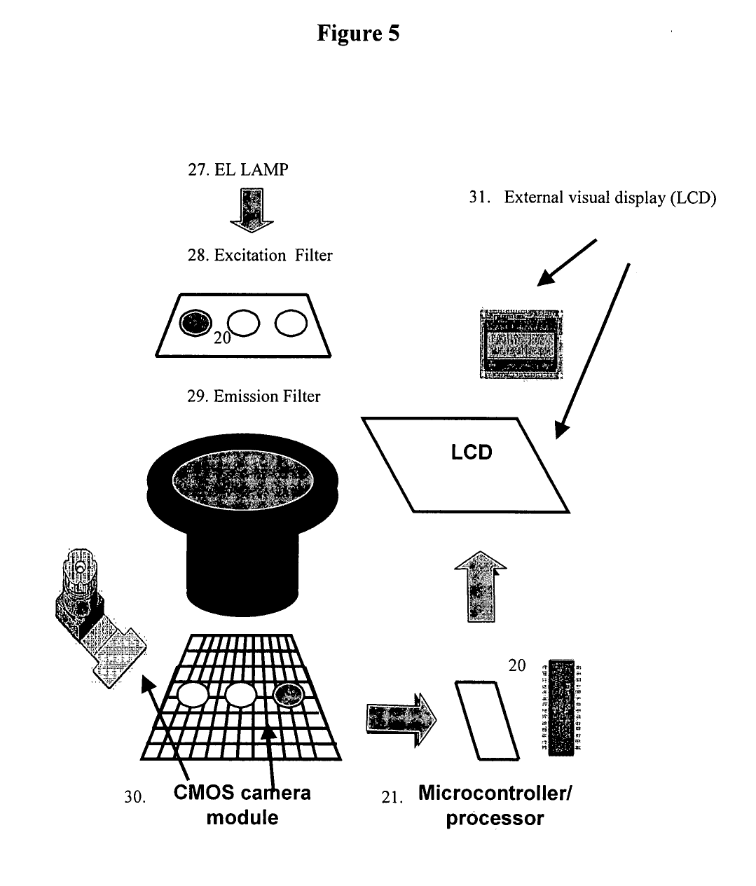

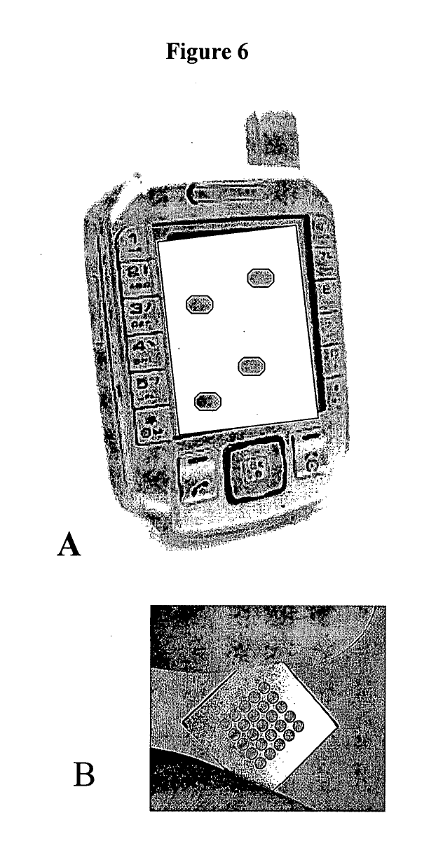

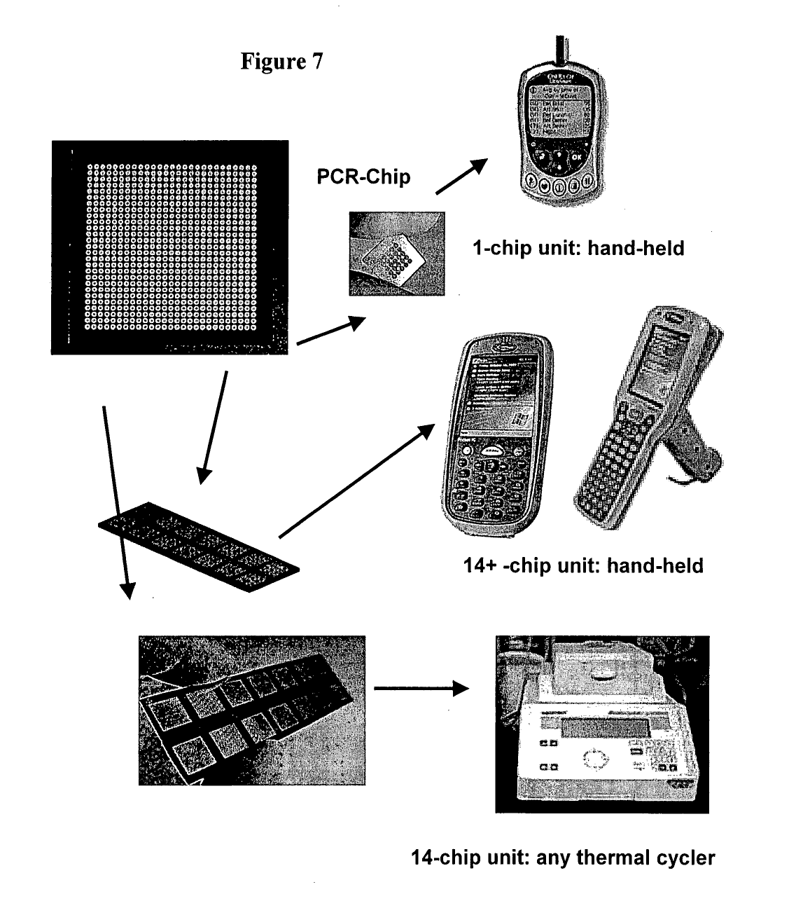

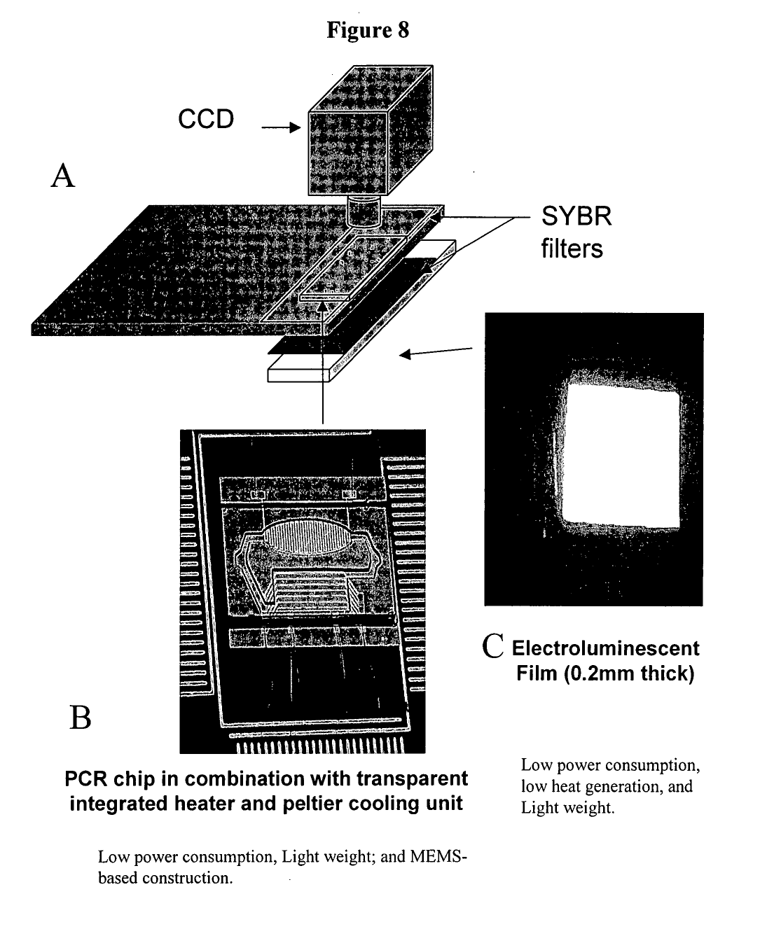

The present invention further encompasses EL-based real-time PCR analyzer devices comprising an EL-based hand-held florescence detection device in combination with components for PCR thermal cycling reactions. FIG. 5 shows an exemplary schematic diagram of the image path of an EL-based hand-held pathogen analyzer of the present invention. Please note that elements in this diagram are not drawn to scale.

The “old” types of portable PCR devices incorporated Peltier units or integrated resistive heaters for thermal cycling of reagents on a solid PCR chip wherein the solid heating elements and the solid chip would inhibit real-time optical detection within the optical path.

In order to overcome such “old” limitations, the inventors contemplate specific types of solutions. In one embodiment, the PCR thermal cycling elements or units are in optical connection with the ELF light source and the sample well. Thus, optically connected heating units, cooling units and sample wells would be optically transparent to the electroluminescent light pathway for allowing real-time or end fluorescent measurements. Therefore, three types of solutions are contemplated. The first is using a transparent heater, such as those described below, in combination with a transparent cooling unit, such as a microfluidics based cooling unit, described below, or using a transparent peltier unit in combination with an optically transparent sample well. The second is to provide an integrated heating unit and cooling unit that is not in optical combination, in other words these units would be out of the optical path so as not to impede fluorescent signal detection. An integrated heating unit and cooling unit would further comprise an optically transparent sample well and electronics that would allow the movement of the samples and/or sample well between the heating/cooling area and the optical path of the ELF source for measuring fluorescence of the biological sample, as described below.

Finally, the inventors contemplate an ELF-based hand-held PCR analyzer for isothermal PCR assays. In one embodiment, an isothermal PCR Analyzer of the present invention would not comprise a microreactor or a thermal cycling unit. In one embodiment, an isothermal PCR Analyzer of the present invention would comprise a thermal cycling unit.

In any embodiment, a heating unit would be capable of heating a sample to the desired temperature for a PCR or isothermal PCR assay.

A. Heating Units and Methods of Use.

The type of heating elements comprising a heating unit would match the configuration of the ELF-based PCR analyzer of the present invention. The inventors contemplate incorporating integrated heating elements in the devices of the present inventions. Heating elements drive the increase in temperature for PCR reactions. The inventors do not intend to limit the type of heating element for use in the devices of the present inventions. Indeed, several types of heating elements are contemplated. In one embodiment, the inventors contemplate an integrated transparent heater. In one embodiment, the analyzer would comprise a stationary sample holder such that the heater is a transparent heating element in optical combination with the sample wells. In another embodiment, the analyzer would comprise a moving sample holder, such that the heating unit would be an opaque heating unit or opaque miniaturized thermal cycler in operable combination with a cooling unit. Further, the heating unit would be out of the optical path so as not to impede fluorescent signal detection while the samples would be moved into and out of the optical path as desired.

B. Transparent Heating Units and Methods of Use.

In one contemplated embodiment, the invention provides an EL film (ELF) based PCR analyzer device for microbial detection comprising a miniaturized thermocycler comprising a transparent heater. In one embodiment, the position of the heating element creates an optical path for providing real time fluorescent detection of DNA. In one embodiment, the CMOS image sensor chip between the heating element and the PCR-chip. In one embodiment, the transparent heater will be placed in between the electroluminescent emitting film/emission filter and the PCR chip. In one embodiment, the transparent heater is at least 4 inches in diameter. In one embodiment, the transparent heater is at least 3 inches in diameter. In one embodiment, the transparent heater is at least 2 inches in diameter. In one embodiment, the transparent heater is at least 1 inch in diameter.

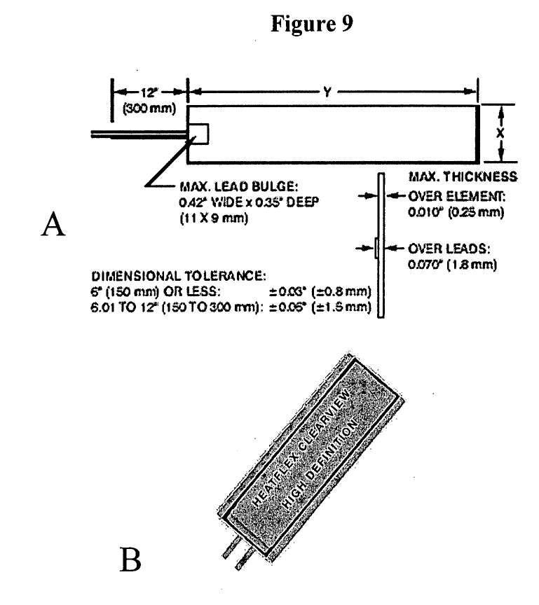

The inventors contemplate using one of at least two types of components to overcome optical and size limitations for providing thermal cycling heaters of the PCR analyzers of the present inventions. First, the inventors contemplate using transparent heaters. An example of such a transparent heater would comprise a micro-thin heating wire laid in between optical grade polyester sheets, which will not only provide uniform temperature distribution but also transmit light. These heaters will be placed in between electroluminescent back-light and the PCR chip, thus providing real time detection of fluorescence with minimal infringement by the heaters. An example of such a transparent heater is a Thermal-Clear Transparent Heater (Minco Worldwide Headquarters) (see, Minco Bulletin HS-202(D)), based on resistive heating that can reach a temperature of 120 degree C. while 80%-90% optically transparent. Another example of such a transparent heater is a Heatflex Clearview Heater (Heatron), that comprises an ultra fine wire (<0.0009 diameter) and thin laminated construction (0.006-0.010 inches thick >90% light transmission. Further this heater is available with integrated transparent Resistance Temperature Detectors (RTD) sensors that measure temperature by correlating the resistance of the RTD element with temperature. FIG. 9 shows an exemplary schematic of EL-Based PCR-chip analyzer heating components.

C. Cooling Units, Microfluidics, and Methods of Use.

Polymerase chain reactions require cooling samples in between heating cycles for optimal thermal cycling. The inventors contemplate a variety of cooling means including transparent or opaque units. Thus, the PCR Analyzer device of the present inventions further comprises a cooling unit, for example, a peltier unit or a microfluidics based cooling unit. In one embodiment, the cooling unit is transparent to light. Such an optically transparent unit may provide fluidics based or air-based (fan) or peltier-based cooling of the samples. Examples of miniature fluidics systems are provided; U.S. Pat. Nos. 5,304,487; 5,922,591; U.S. Patent Appln. Nos. 20030091476; 20030118486; and 20060188413; all of which are herein incorporated by reference.

In a further embodiment, the opaque cooling unit comprises a heating unit. The inventors contemplate that following a cycle of heating and cooling, the sample is transported into the optical path wherein the fluorescence is measured as described herein, then returned if another round of heating and cooling is desired.

D. Miniaturized Thermal Cycler Units and Methods of Use.

In one contemplated embodiment, the invention provides an EL film (ELF) based PCR analyzer device for microbial detection comprising a miniaturized thermal cycler unit. In one embodiment, the thermal cycler unit in located within the Hand-held device for providing standard PCR using a transparent sample holder. Upon completion of the PCR amplification, the sample holder is transported to the optical path for providing a measurement of incorporated fluorescence. For those reactions that necessitate removing unincorporated markers/dyes, the hand-held device further comprises compositions and methods for removing unincorporated fluorophores. Further, examples of miniaturized reactors and more specifically miniaturized amplification reactors and methods for microchip-based reactions useful to the present ELF based devices of the present inventions are provided in the following publications: U.S. Pat. Nos. 5,498,392; 5,587,128; 5,639,423; 5,674,742; 5,646,039; 5,786,182; 6,261,431; 6,432,695; and 6,126,804; German Patent No. DE 4435107C1; and Xiang et al., (2005) Biomedical Microdevices, 7(4):273-279(7); all of which are herein incorporated by reference.

E. Isothermal Amplification.

The inventors contemplate an ELF based hand-held PCR analyzer device for providing isothermal nucleotide amplification and analysis, such that the amplification step proceeds either at one temperature or a narrow temperature range, such as at 64° C. or ranging in temperature from 37° C. to 65° C. In other words, isothermal amplification does not require a standard thermal cycling device for cycling between temperatures such as between 45° C. to 95° C., such that temperatures of 45° C. to 60° C. for primer annealing, 95° C. for double-stranded separation, with amplification at 72° C. The inventors contemplate chemical or molecular mediated disassociation of DNA strands and DNA polymerase and/or RT that functions at room temperature or a specific desired temperature. Examples of compositions and methods of isothermal amplification include but are not limited to using a thermophilic Helicase-Dependent Amplification (tHDA) method, such as an IsoAmp tHDA kit (BioHelix Corp.). Similar to PCR amplification, a tHDA reaction selectively amplifies a target sequence defined by two primers. However, unlike PCR, tHDA uses a helicase enzyme to separate double-stranded DNA, rather than heat. Thus DNA can be amplified at a single temperature without the need for thermal cycling or without a need for more than one cycle of heating and cooling. Isothermal amplification may take place at 62° C.-65° C., preferably 64° C., primer annealing may take place at 60° C.-80° C.; optimum equals 68-72° C. In one embodiment, the sample chamber with samples is heat denatured for two-three minutes at 95° C. at the beginning of the amplification reaction may enhance performance, then cooled to 0° C. prior to incubation at 62° C.-65° C. Such denaturation can take place either separately from the Hand-held device prior to inserting sample or within such devices capable of at least one cycle of heating and cooling. A further example of isothermal amplification is using an isothermal DNA Polymerase, such as obtained from a cloned gene 2 of Bacillus subtilis phage phi29 DNA Polymerase (Fermentas Inc.). Examples of methods of such isothermal reactions for use with devices of the present inventions as shown in but not limited to Blanco, et al., (1989) J. Biol. Chem., 264:8935-8940; Garmendia, et al., (1992) J. Biol. Chem., 267:2594-2599; Esteban, et al., (1993) J. Biol. Chem., 268(4):2719-2726; all of which are herein incorporated by reference in their entirety, and further include assays, in particular for identifying pathogens such as Escherichia coli O157:H7, as in Loop-mediated isothermal amplification (LAMP) assays, as described in Vora, et al., (2004) Appl Environ Microbiol. 70(5):3047-54; herein incorporated by reference, and additional methods as in Vincent et al., (2004) EMBO reports 5(8):795-800; and Barker, et al., (2005) BMC Genomics, 22;6(1):57; all of which are herein incorporated by reference and real-time isothermal DNA amplification, such as Rolling-circle amplification (RCA) and ramification amplification (RAM, also known as hyperbranched RCA) PCR, for example, Yi, et al., Published online 2006, Nucleic Acids Research 2006 34(11):e81; herein incorporated by reference.

F. PCR Pizza Wheel Sample Reaction Chamber.

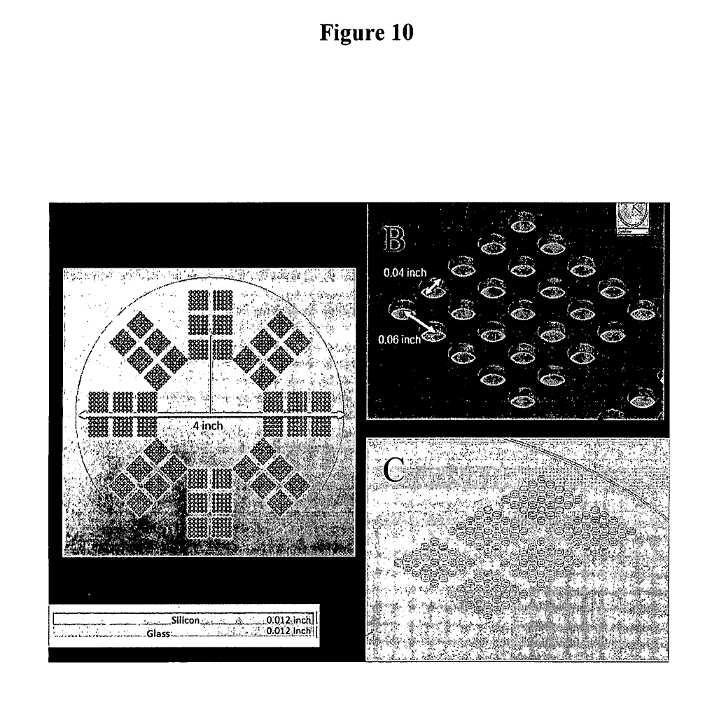

Another component contemplated by the inventors is a transparent reaction chamber mounted on a Pizza Wheel chip or Pizza Wheel wafer for use in the devices of the present inventions. In an exemplary schematic, the inventors contemplate a 4-inch chip or wafer as drawn with CAD software, FIG. 10, however a chip may be any size capable of being used in the devices of the present inventions. In one embodiment, said chip may be used in conventional PCR devices for analysis in ELF based detection devices of the present inventions while alternatively, the chip may be used for PCR assays within an ELF-based PCR analyzer of the present inventions. The Pizza Wheel chip may comprise silicon wells and/or Polydimethylsiloxane (PDMS), such as replica molding described in Sia and Whitesides, (2003) Electrophoresis, 24:3563-3576, and/or silicone and glass (BioTrove); all of which are herein incorporated by reference. A quality of PDMS particularly useful to the present invention is transparency to light.

Even further, the inventors contemplate using on-chip PCR reactions in transparent reaction chambers of the chip. Thus allowing through chip optical detection during real-time PCR reactions. For one example of a transparent PCR reaction chamber, see, BioTroves' Through hole microwell plates used with conventional and real-time bench-Top PCR devices. Each assay requires approximately 33 nanoliter. The inventors contemplate the use of 0.04 inch (1.016 mm) sample wells, such as shown in FIG. 10.

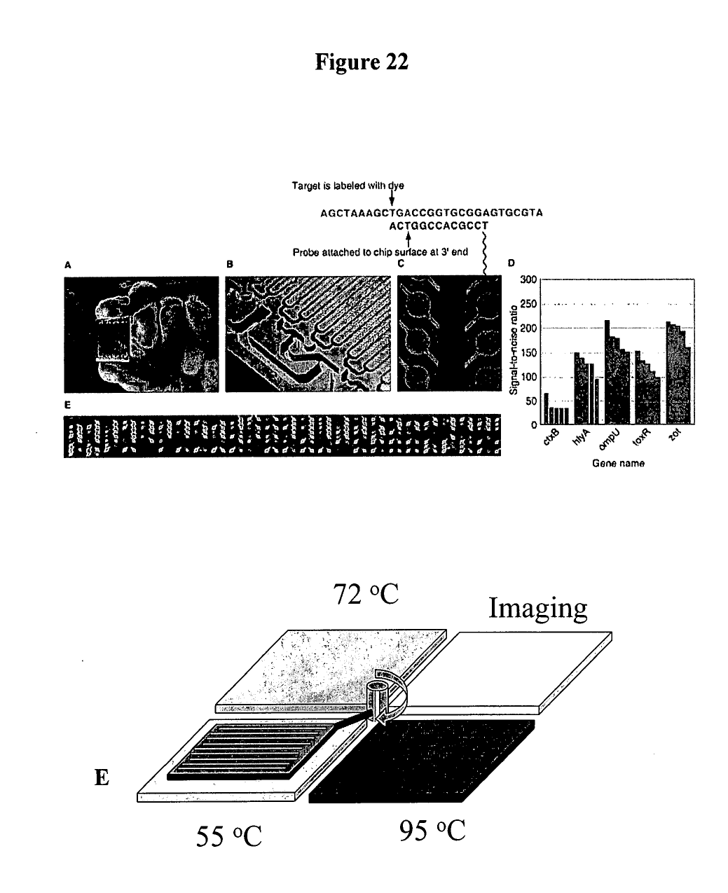

In one embodiment, the inventors contemplate a stable pizza wheel chip, such that once the chamber is in place it is not moved between cycles, such as for use with transparent heaters and cooling units or for isothermal reactions, thus remaining in the optical path of the ELF light source. In one embodiment, the inventors contemplate a moveable pizza wheel chip that is capable of being moved electronically and/or/mechanically within the hand-held device, such as for use with non-transparent microreaction units. In one embodiment, the transparent reaction chip is a disposable (one time use) reaction chamber. In one embodiment, the transparent reaction chip is a reusable reaction chip. In one embodiment, the transparent reaction chip remains intact during high temperature and cooling cycles of PCR thermal cycling. In one embodiment, the transparent reaction chip is capable of being used with isothermal reactions, such as those described herein. In one embodiment, the inventors contemplate moving the chip while the heaters remain in one place, in this case the heaters may have solid components (FIG. 22F)

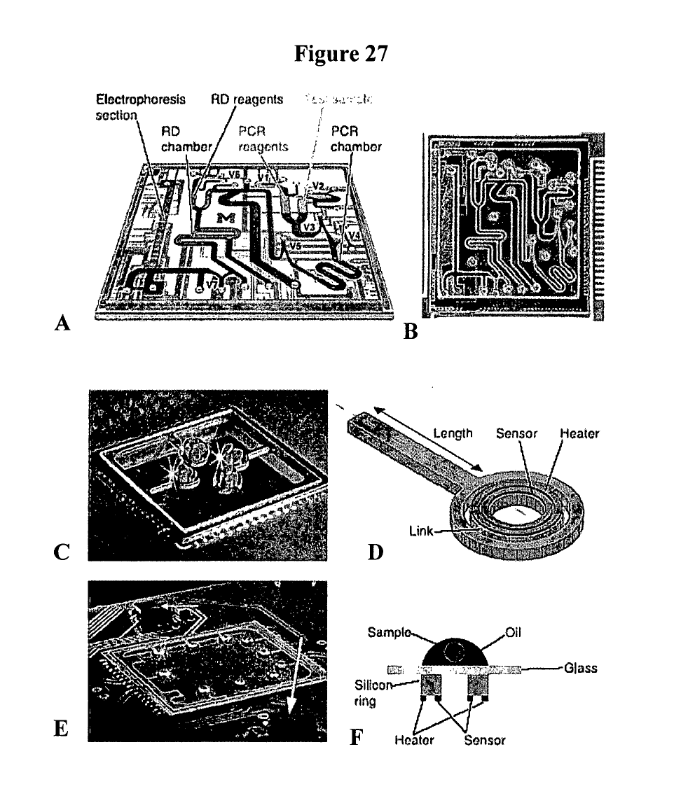

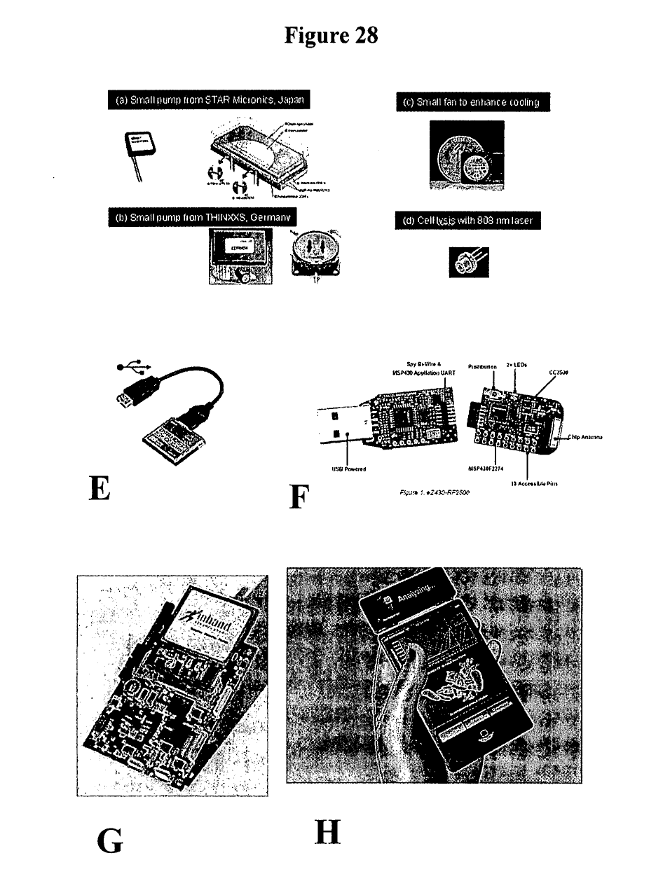

The inventors contemplate ELF based PCR hand-held analyzer devices of the present inventions further comprising micromotors for moving chips within the devices of the present inventions, including moving a pizza wheel type chip. Examples of such devices include but are not limited to a miniature/MEMS micromotor or an ultrasonic motor (FLEXMOTOR, flexmotor.com), see, FIGS. 27 and 28.

### IV. Methods Relating to Using EL-Based Fluorescent Detectors and Analyzers of the Present Inventions.

A. Types of Fluorescent Labels.

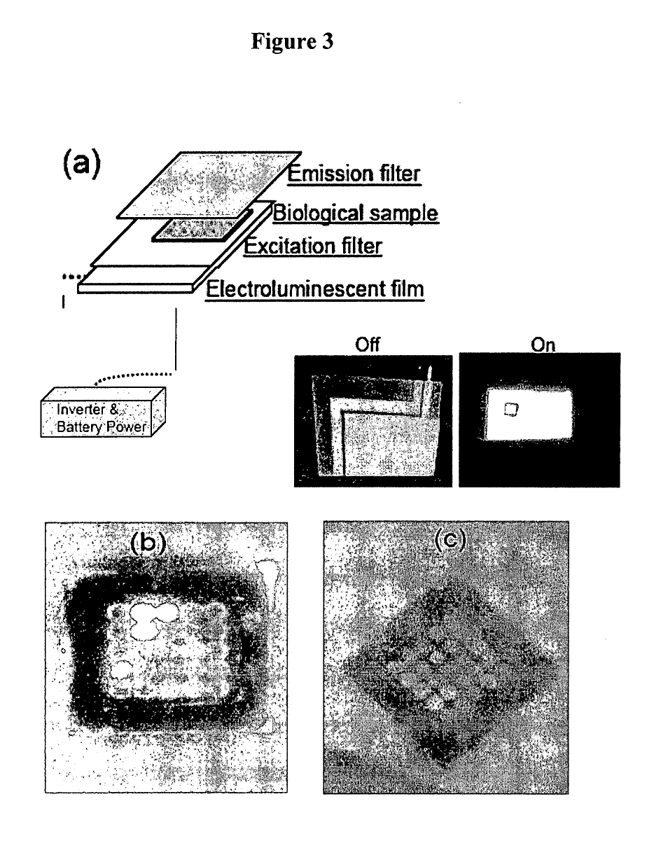

The inventors successfully tested a blue light ELF illumination of a fluorescenct biological sample, for example, amplified DNA with and without incorporated SYBR™ Green fluorescent compound in combination with a SYBR™ Green compatible set of excitation and emission filters, see, FIGS. 3b and 3c. Thus the inventors further contemplate using a variety of combinations of ELF excitation, fluorescent compound and compatible filters in the detection devices of the present inventions.

In particular, the inventors contemplate the use of ELF emitting devices chosen from the group consisting of blue, green, read and yellow EL emitting films.

The inventors contemplate the use of numerous types of fluorophores, fluorescent compounds, and fluorescent dyes. In one embodiment, said fluorescent compound is selected from the group consisting of SYBR™ Brillant Green, SYBR™ Green I, SYBR™ Green II, SYBR™ gold, SYBR™ safe, EvaGreen™, a green fluorescent protein (GFP), fluorescein, ethidium bromide (EtBr), thiazole orange (TO), oxazole yellow (YO), thiarole orange (TOTO), oxazole yellow homodimer YOYO, oxazole yellow homodimer YOYO-1, and derivatives thereof.

The devices of the present invention are contemplated to differentiate between different dyes using instrumental methods, for example, a variety of filters and diffraction gratings may be employed (e.g. to allow the respective emission maxima to be independently detected), in addition to appropriate compatible software. When two dyes are selected that possess similar emission maxima, instrumental discrimination can be enhanced by insuring that both dyes' emission spectra have similar integrated amplitudes, similar bandwidths, and further by insuring that the instrumental system's optical throughput is equivalent across the emission range of the two dyes. Instrumental discrimination can also be enhanced by selecting dyes with narrow bandwidths rather than broad bandwidths, for example, detection methods are provided in International publication No. WO9853093; herein incorporated by reference.

Fluorescent staining of sample particles, such as DNA, may be achieved by any of the technique known in the art, examples of making fluorescent particles include: (i) covalent attachment of dyes onto the surface of the particle (e.g. U.S. Pat. No. 5,194,300; herein incorporated by reference), (ii) internal incorporation of dyes during particle polymerization (e.g.; U.S. Pat. No. 5,073,498; herein incorporated by reference), and (iii) dyeing after the particle has already been polymerized.

Fluorescence detection systems (including visual inspection) are used to detect differences in spectral properties between dyes, with differing levels of sensitivity. Such differences include, but are not limited to, a difference in excitation maxima, emission maxima, fluorescence lifetimes, fluorescence emission intensity at the same excitation wavelength or at a different wavelength, a difference in absorptivity, a difference in fluorescence polarization, a difference in fluorescence enhancement in combination with target materials, or combinations thereof.

B. Types of Chips.

The inventors contemplate a variety of PCR chips for use with the devices of the present inventions. In particular, the sample chambers allow the passage of EL light emissions for providing a fluorescent signal corresponding in intensity to the concentration of fluorophore incorporated into the biological sample. In one embodiment, the PCR chip is processed in a conventional PCR machine and then inserted into an EL Fluorescent detector of the present invention.

In one embodiment, the EL-based detector and PCR analyzer of the present invention provides information using a chip or microarray with an optically transparent sample chamber. One example of an optically transparent sample chamber is provided using PDMS, wherein the entire chip is optically transparent. Another example is provided using glass and silica, wherein the sample well is optically transparent through the glass bottom, or an optically equivalent of glass, while the sides of the wells and the remainder comprise silica).

In one embodiment, the inventors contemplate a sample chamber 300 μm in diameter with a depth of 300 μm with no solid base or top, where liquid is held in place by surface tension. In one embodiment, a sample chamber, as shown in FIG. 10, holds 33-nl of fluid. In one embodiment, the surface of the sample chamber is hydrophobic, while rendering the interior of the hydrophilic and biocompatible, an example of such a well is provided by an OpenArray™ plate (BioTrove).

In another embodiment, the inventors contemplate using On-Chip PCR reactions for PCR analysis using an EL based PCR analyzer device of the present inventions. The inventors contemplate on-chip amplification using chips, such as a transparent chip, an open-hole pizza wheel chip, and any chip compatible with a device of the present inventions.

In one embodiment, such chips would comprise on-chip oligonucleotide primers for PCR amplification. Methods for providing on-chip primers would be compatible with the chips used by the ELF based PCR analyzer devices, and would include dispensed or attached primers. Dispensed fluids are in the micro to nanoliter range. Methods for providing dispensed primers are based upon robotics mechanisms and would comprise dispensing pre-synthesized primers, such as provided in a “whole chip” sleeve for dispensing into a chip, or a combination of synthesizing primer pairs then dispensing into wells, such as into wells of a 96 well plate or sample spots or wells of chips. For example, primer dispensing into low-density chips would be manual or by hand-held pipetter or small machine for dispensing primer sets. In one embodiment, the primers are dispensed into each sample chamber, then lyophilize for adhering primers to chamber, wherein the primers would be released upon contact with fluid. In one embodiment, a dispensing mechanism is used for dispensing primers into sample chambers. In a further embodiment, said dispensing mechanism is used for dispensing buffer, DNA polymerase plus reaction components with or without primer and with or without sample. Examples of such a dispenser mechanism are described in U.S. Patent Appln. No. 2003175163 and U.S. Pat. No. 6,079,283; all of which are herein incorporated by reference.

The inventors contemplate a “hook” method for providing on-chip primers, wherein said primers would release upon the first heating cycle of a PCR reaction. Examples of such primers are shown in FIG. 11. These on-chip primers would be double-stranded DNA oligonucleotides wherein one strand, the “hook” would be attached to the chip while the other complementary strand would be released from the chip upon reaching the melting temperature of the oligonucleotide or being contacted with a denaturation chemical/molecule. Following on-chip hook synthesis, samples and reaction components would be injected under cold temperatures, using microfluidic channels such as those described herein.

Each oligonucleotide hook will be synthesized on-chip using any one of a variety of methods, including but not limited to a liquid phase phosphoramidite chemistry reaction, for examples, see, U.S. Pat. No. 6,426,184; and U.S. Patent Appln. Nos. 20020081582; 20030138363; 20030143131; 20030186427; and 20040023368; all of which are herein incorporated by reference. Briefly, a phosphoramidite-based technique will build a DNA oligonucleotide sequence, one nucleotide at a time, attached by a 5′ nucleotide to the chip. This technique uses a photo acid precursor (PGA) that becomes a strong acid when exposed to light directed with a digital micromirror device (DMD). The strong acid is generated directly at the point of synthesis, where a nucleotide is isolated and protected from addition of new nucleotides with a protection molecule. The acid removes the protection molecule, and allows the next nucleotide and protection molecule to bond to their proper place the sequence. In this manner, sequences greater than 100 base pairs can be synthesized. The technique is cost effective because of using DMD, thus traditionally used and expensive photolithographic masks would not be required. However, in other embodiments, primers and/or hooks would be prepared off-chip for using microfluidics to wash primers and/or hooks into sample wells/chambers. For example, for high-density PCR chips, hooks would be synthesized on one chip, while primers are synthesized on a different chip. In one embodiment, each well would comprise at least one sequence of a 9-10 mer hook and a specific primer. Thus samples would be analyzed in one of several ways. In one embodiment, wherein each well would comprise one type of sequence of a primer/hook, one RNA and/or DNA sample would be added to the wells. In another embodiment, wherein each well would comprise a different RNA and/or DNA sample. In another embodiment, the inventors contemplate a DNA primer printer for a microarry chip. Thus printing a primer on a flat surface, then build sample wells around the primer using polydimethysiloxane (PDMS).

C. Types of Samples and Reagents for On-Chip RT-PCR: EL Based Hand-Held PCR Analyzer.

The inventors contemplate PCR chips comprising on-chip samples and reagents. In one embodiment, on chip samples and reagents are added to a PCR chip prior to loading the PCR chip into an EL-based PCR analyzer device of the present invention. In one embodiment, a PCR chip comprising appropriate samples and reagents is inserted into a PCR analyzer of the present invention for a conventional PCR, such as a RT-PCR. In another embodiment, a PCR chip comprising appropriate PCR samples and reagents is inserted into a PCR analyzer of the present invention for a real-time PCR, such as a qPCR. In one embodiment, the PCR chip comprises, primers, and a DNA sample, such as a microbial DNA target, and PCR reagents. In a preferred embodiment, a PCR chip for insertion into an EL-based PCR analyzer device of the present invention comprises a DNA sample, such as a microbial DNA sample.

Types of preloaded PCR reagents include but are not limited to DNA polymerase, such as a Taq DNA polymerase, dNTPs, a reaction buffer, such as Hepes, PCR grade water, and a salt, such as MgCl₂. Additionally, reagents may also comprise, M-MuLV Reverse Transcriptase, an RNase Inhibitor, etc. Examples of preloaded reagents include but are not limited to a lyophilized reagent, a freeze-dried reagent and the like.

Specifically, the inventors contemplate pre-dispensed reagents for PCR analysis using an EL Based Hand-held PCR Analyzer device of the present inventions. Examples of such pre-dispensed reagents include PuReTaq Ready-To-Go™ PCR Beads (Amersham Biosciences), Ready-To-Go™ RT-PCR Beads (Amersham Biosciences), SmartMix™ HM MasterMix bead for either a single-target or a multiplexed real-time PCR reaction (Cepheid) and the like. Examples of pre-dispensed reagents include but are not limited to a lyophilized reagent, a freeze-dried reagent and the like.

### V. Economic Feasibility.

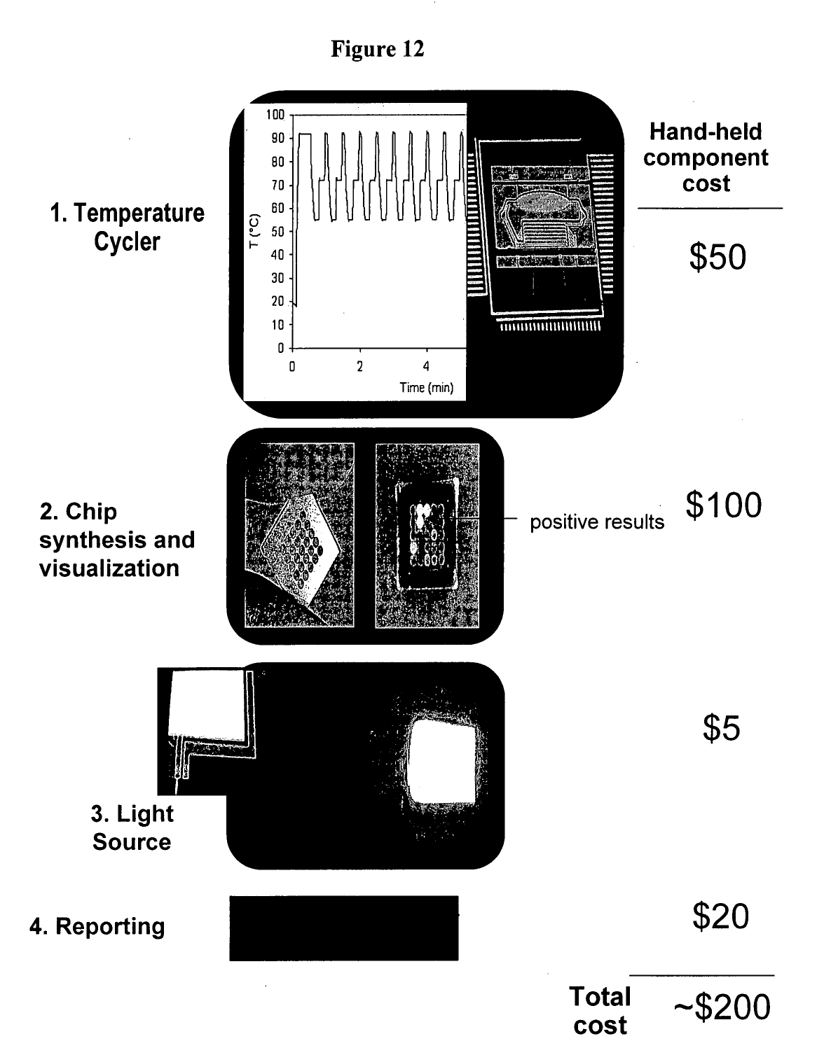

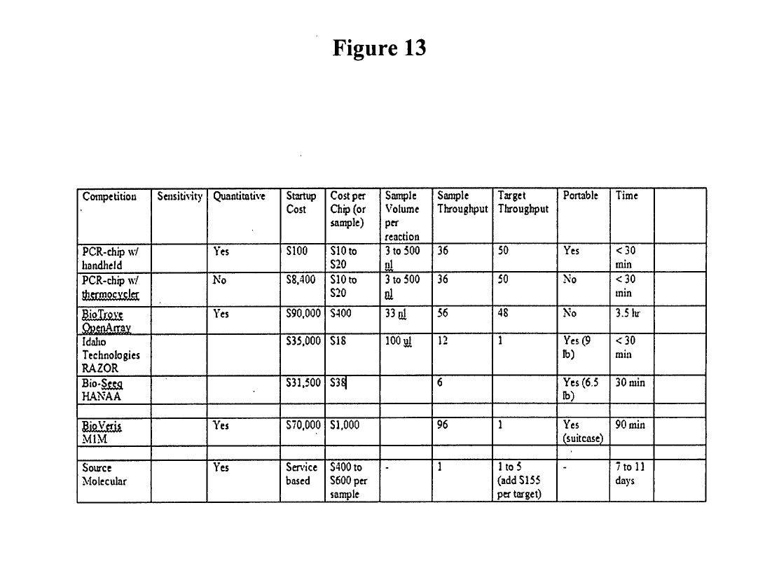

The inventors provided cost estimates for the major components to provide fluorescent detection devices and analyzers of the present inventions. For a cost, weight, cost per sample and number of samples per run comparison between PCR devices, see, FIGS. 13-16. The inventors initially provide an exemplary cost estimate for providing a simple ELF based detection assay, including a basic Hand-held of the present invention, on-chip synthesis, visualization with an ELF incorporated in the hand-held, and recording of information. See, FIG. 12. Further, the inventors provided cost estimates for providing chips for on-chip PCR for use in the fluorescent detection devices of the present inventions. In particular, unlike the currently available hand-held PCR devices, the hand-held devices of the present inventions are economical and lightweight as opposed to commercially available expensive and heavy PCR devices. The inventors contemplate that a hand-held device of the present invention will comprise components whose total cost is about $1000 U.S. compared to $30-35,000 U.S. fora RAZOR™ or HANAA™. Further, the inventors contemplate that an ELF based device of the present invention will be 1/10 in weight of RAZOR™ or HANAA™ devices and will analyze samples from up to 50 pathogens per sample run. Further, in combination with primer sets developed by the inventors, in particular for a virulence-marker gene (VMG) chip for 20 major human pathogens, the analysis should be more complete and economical than from currently available assays. FIG. 12 illustrates exemplary embodiments, showing the wells, the temperature cycling, and how the positive results can be visualized, all with components that costs less than or equal to $200 (U.S.).

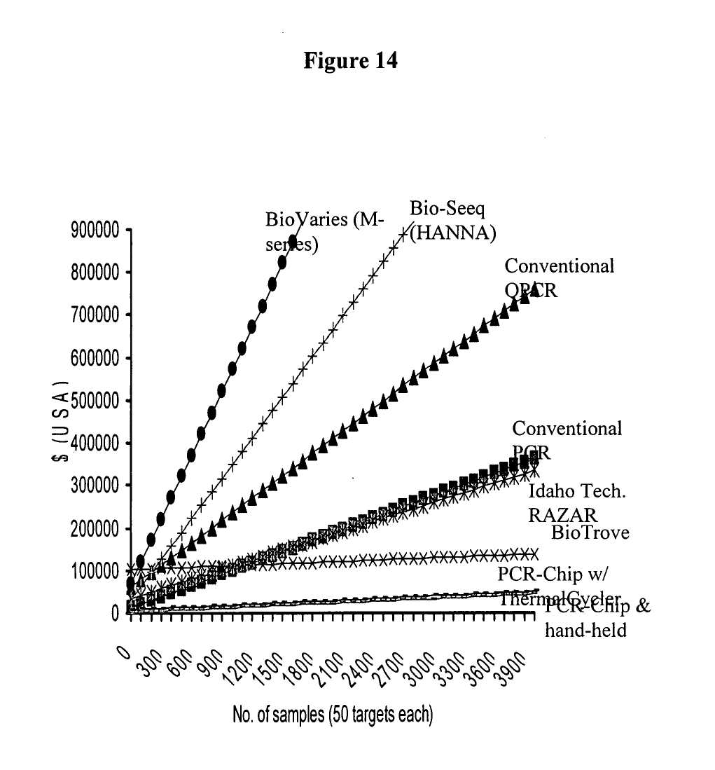

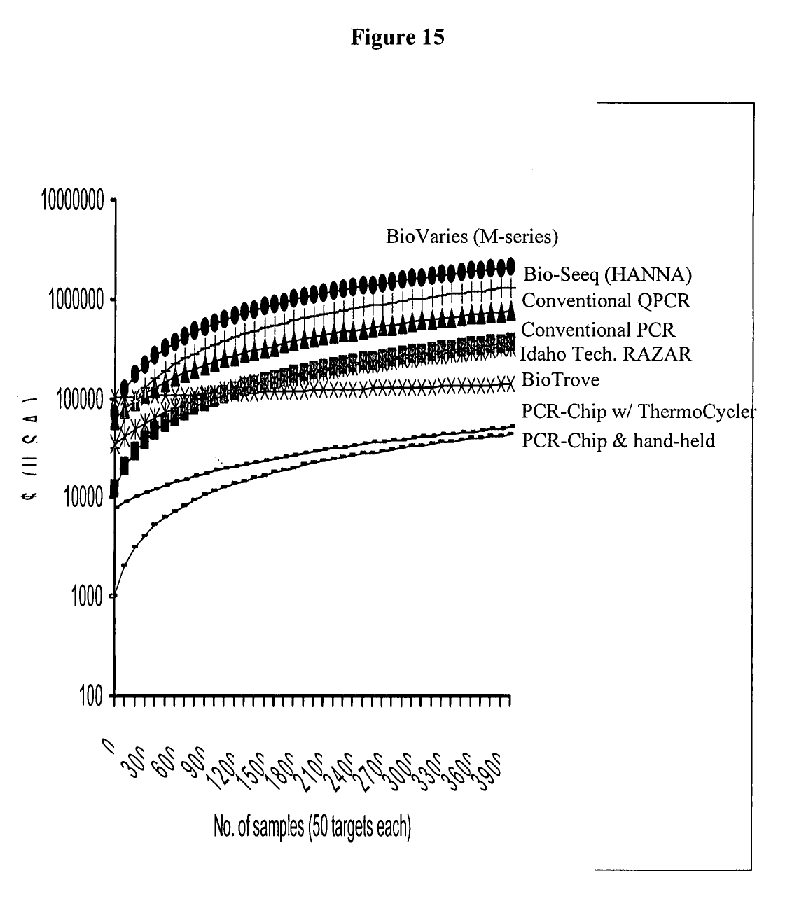

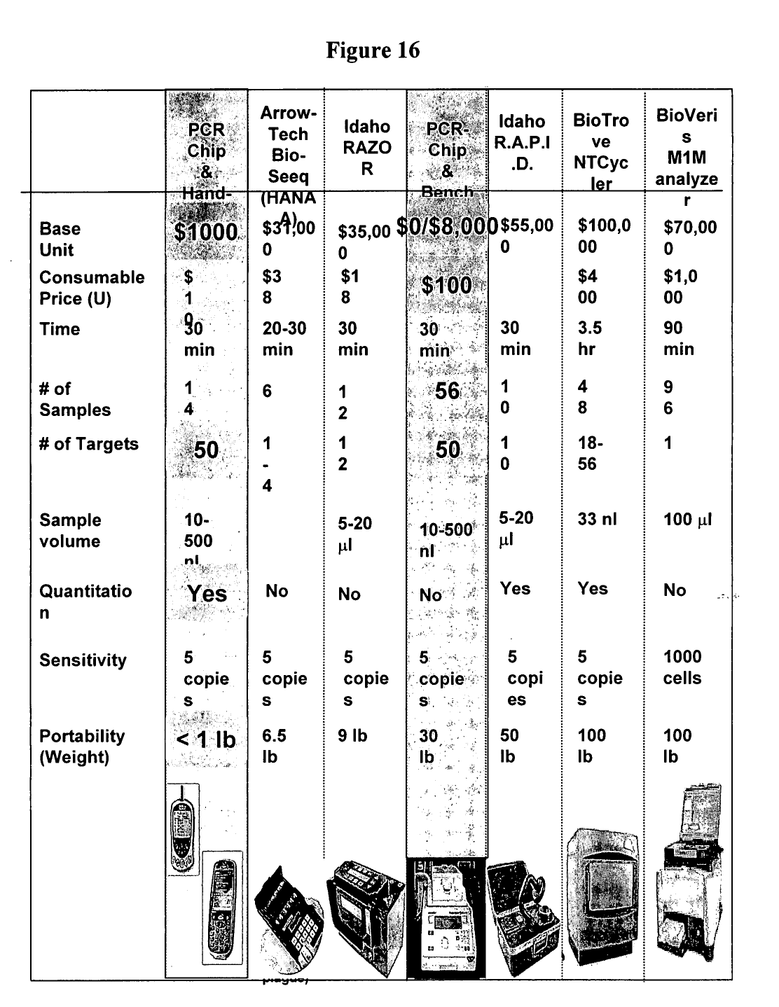

The inventors further contemplate that a multi-sample PCR-chip such as those described herein, have the potential to become a leading consumable product in labs that already have a thermalcycler because it will reduce the cost substantially. The inventors contemplate cost per sample of less than HANAA™ and equal to or less than RAZOR™, for examples, see, FIGS. 13-15. Further, the inventors contemplate start-up cost per sample run, including reagents and primers. Thus, FIGS. 14 and 15 show an exemplary direct and semi-log scale comparison, respectively, of cost per sample between PCR Chip & EL-Based Bench-Top and PCR Chip & EL-Based Hand-held Pathogen Analyzer and commercially available devices, such as the RAZOR™ and the HANAA™. The inventors further show in FIG. 16 overall comparisons of contemplated superior PCR Chip & EL-Based Bench-Top and EL-Based Hand-held Pathogen Analyzer to commercially available devices demonstrating the economic feasibility of providing and using the contemplated devices of the present inventions.

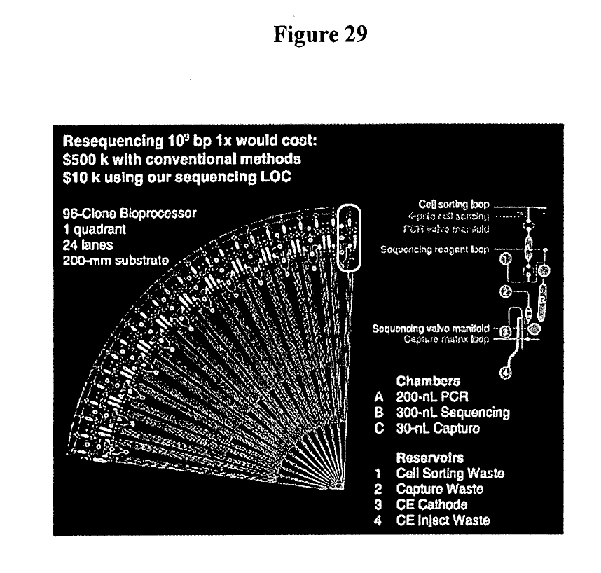

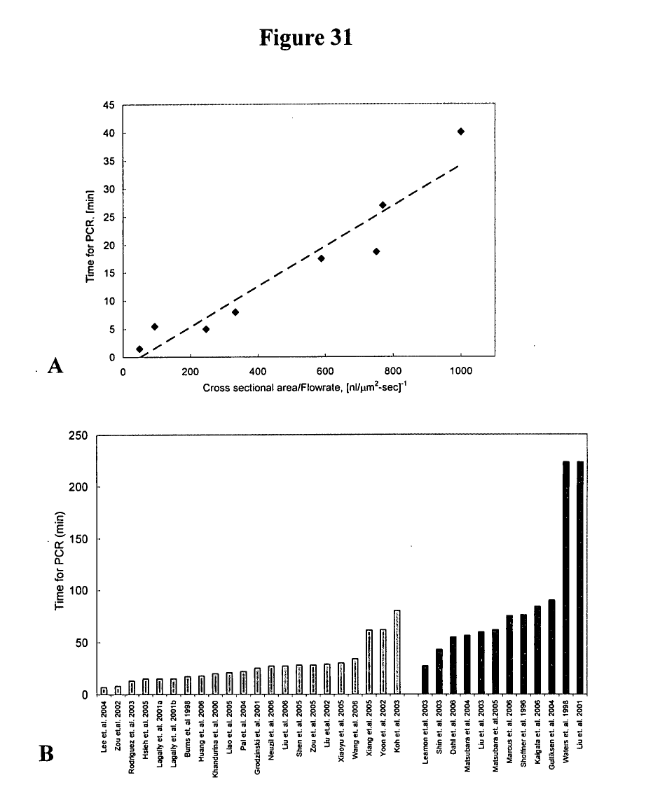

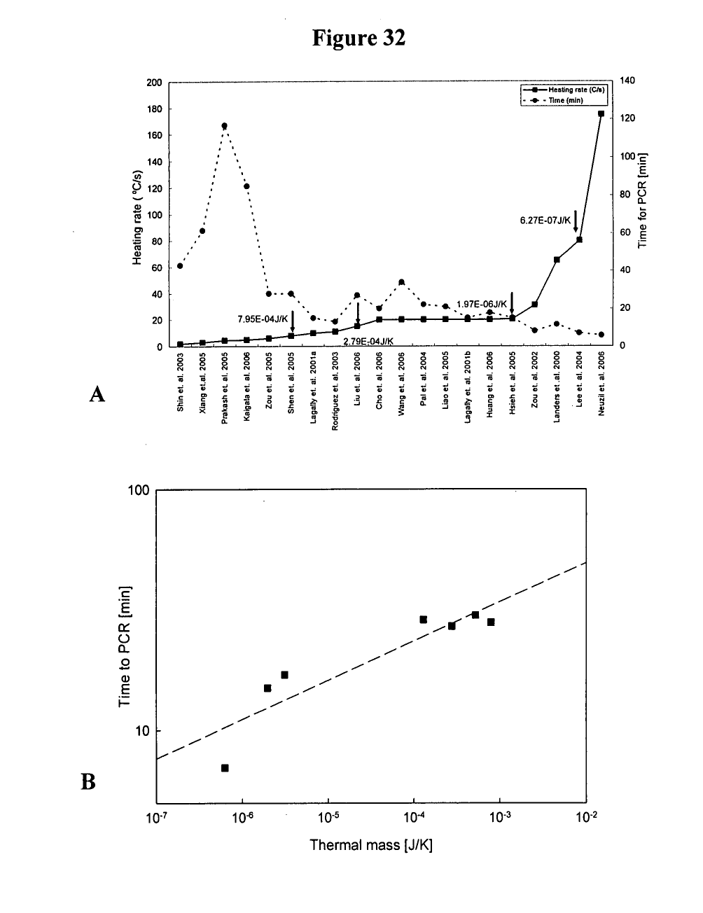

The inventors further provide an exemplary analysis of literature for static, integrated heater, and Flow-through microPCR Chips (FIGS. 31 and 32 and Tables 2-4. Including an example of a Highly parallel sequencing on a wafer for reducing the cost of resequencing and SNP detection significantly in a clinical setting (FIG. 29).

TABLE 2
The important parameters of continuous flow PCR system studies used for
theoretical analysis in FIG. 31A.

| | | | | | | | Sadler | |
|---|---|---|---|---|---|---|---|---|
| Factors/ | Kopp et al. | Obeid et al. | Park et | Hashimoto et | Schneegaβ | Hashimoto | et al. | Chou et al. |
| References | 1998 | 2003 | al. 2003 | al. 2004 | et al. 2001 | et al. 2006 | 2003 | 2002 |
| Time of | 1.5 | 5 | 5.5 | 8.6 | 17.5 | 18.7 | 27 | 40 |
| amplification | | | | | | | | |
| (min) | | | | | | | | |
| Number of | 20 | 20 | 33 | 20 | 25 | 30 | 40 | 30 |
| cycles | | | | | | | | |
| Flow rate | 72.9 | 21 | 83.33 | 22.5 | 33 | 6.67 | 325 | 250 |
| (nL/s) | | | | | | | | |
| Cross-sectional | 3600 | 5181 | 7850 | 7500 | 19625 | 5000 | 250000 | 250000 |
| area (μm²) | | | | | | | | |
| Channel | 2.2 | 3.43 | 3.5 | ???? | 1.512 | 1.57 | | — |
| length (m) | | | | | | | | |
| Volume of | 10000 | 7600 | 50000 | ???? | 33000 | 168 | 24000 | 19000 |
| fluid (nL) | | | | | | | | |
| Fluid delivery | Syringe pump | Syringe | Syringe | Syringe pump | Syringe | Syringe | Peristaltic | Peristaltic |
| | | pump | pump | | pump | pump | pump | pump |
| Target copies | 1 × 10¹ | 2.5 × 10⁶- | — | 2 × 10⁷-1 × 10⁸ | — | — | — | — |
| | | 1.6 × 10⁸ | | | | | | |
| Material | glass/serpentine | borosilicate | Fused | Polycarbonate | Glass/ | Polycarbonate | LTCC/ | LTCC/ |
| (chip)/Design | | glass/serpentine | silica | (PC)/spiral | serpentine | (PC)/serpentine | serpentine | serpentine |
| | | | capillary | loops | | | | |
| | | | coils/helical | | | | | |
| Material | Copper blocks | Copper | Copper | Resistive | Platinum | Film | Ag—Pd | Screen |
| (heater) | | blocks | blocks | heaters | thin film on | resistance | thin film | printed Ag/Pd |
| | | | | | silicon | heaters | | paste |
| Temperature | PID digital | PID digital | Manual | Closed loop | Analog | Closed loop | PI | Not |
| Control | temperature | temperature | | PID controller | electronic | PID | controller | mentioned |
| | controller | controller | | | controller | controller | | |
| Process | Not done | Not done | Not done | ANSYS/CFD- | Not done | Not done | CFDRC- | CFDRC- |
| simulation | | | | FLOTRAN | | | ACE+ | ACE+ |
| Surface | Dichlorodimethyl | Dichloro- | Trimethyl | Bovine serum | Hexamethyl | No treatment | Not | Not |
| treatment | silane, dynamic | dimethylsilane, | chlorosilane/ | albumin | disilane/ | | mentioned | mentioned |
| | | static | DMF/imidazole, | (BSA), static | BSA, | | | |
| | | | static | | static and | | | |
| | | | | | dynamic | | | |

*Low temperature co-fired ceramics (LTCC)

TABLE 3
The calculated values of thermal mass of integrated heaters in static PCR
systems

| | | Specific heat | Density | Heater dimensions | Thermal mass |
|---|---|---|---|---|---|
| Reference | Material/Heater | (J/K · g) | (g/cm3) | (μm3) | (J/K) |
| Lee et al. | Platinum | 0.13 | 21.45 | 1500 × 500 × 0.3 | 6.27E−07 |
| 2004 | | | | | |
| Hsieh et al. | Platinum | 0.13 | 21.45 | π × (1500)² × 0.1 | 1.97E−06 |
| 2005 | | | | | |
| Burns et al. | Gold | 0.13 | 19.3 | 500 × 500 × 5 | 3.13E−06 |
| 1998 | | | | | |
| Liu et al. | Platinum | 0.13 | 21.45 | (5 × 10⁴) × (1 × 10⁴) × 0.2 | 2.79E−04 |
| 2006 | | | | | |
| Shen et al. | Copper | 0.39 | 8.92 | 120000 × 55 × 35 | 7.95E−04 |
| 2005 | | | | | |
| Liu et al. | Tungsten | 0.13 | 19.3 | 40000 × 26000 × 0.05 | 1.30E−04 |
| 2002 | | | | | |
| Xiaoyu et al. | Platinum | 0.13 | 21.45 | 35000 × 18000 × 0.3 | 5.27E−04 |
| 2002 | | | | | |

TABLE 4
A brief information about the numerical simulation tools commonly
used for micro-PCR systems.

| Software | Applications | Company | Reference |
|---|---|---|---|
| ANSYS | | ANSYS Inc. | |
| | | (www.ansys.com) | |
| CFD-RC | | CFD Research Corporation | |
| | | (http://www.cfdrc.com) | |
| CFD-ACE + | | CFD Research Corporation | |
| | | (http://www.cfdrc.com) | |
| COSMOS | | Solid Works Corporation | |
| | | (http://www.solidworks.com) | |
| CoventorWare | | Coventor Inc. | |
| | | (http://wwwl.coventor.com) | |

### Experimental

The following examples are provided in order to demonstrate and further illustrate certain preferred embodiments and aspects of the present invention and are not to be construed as limiting the scope thereof.

In the experimental disclosure which follows, the following abbreviations apply: ° C. (degrees Centigrade); mm (millimeters); nm (nanometers); μ (micrometer); U (units); V (volts); sec (seconds); min(s) (minute/minutes); hr(s) (hour/hours); PCR (polymerase chain reaction); RT-PCR (reverse transcription PCR); hertz (Hz); and W (watts);

### Example I

Off-the shelf inexpensive elements for use in EL based fluorescent detector fabrication are described below.

### Detector Elements:

Electroluminescence (EL) film (ELF). Of the numerous types of commercially available electroluminescence (EL) products, see, FIG. 1, an electroluminescent AC thin-film electroluminescent device (ELD of FIG. 1) were tested. See Table 1. Specifically, a 20×28 cm sheet of commercially available ELF (Novatech Electro-luminescent, Chino, Calif.) ($40 U.S.) comprising a phosphor emitter as depicted in FIG. 2, was cut into the desired spatial area, under 5×7 cm, see, FIG. 3.

TABLE 1
Components: source, cost, and spectral specifications.

| | | Maximum |
|---|---|---|
| | | Spectral |
| | | luminance |
| Company | Cost (U.S. $/in²) | (footlambert) |
| E-Lite Technologies, Inc. | $0.46 | 24 |
| 2285 Reservoir Ave. | | |
| Trumbull, CT 06611 | | |
| Electric Vinyl Inc. | $0.60 | 550 (lux) |
| 349 Hidden Lake Road | | |
| Enderby, BC | | |
| V0E-1V0 CANADA | | |
| KNEMA, LLC. | $0.52 | 24 |
| Luminous Film | | |
| 7100 West Park Road | | |
| Shreveport, Louisiana | | |
| 71129 | | |
| *Novatech Electro- | $0.46 | 73 |
| luminescent | | |
| 4821 Lanier Road | | |
| Chino, CA 91710 | | |

*EL film used for initial evaluations.

Electronic Wiring of ELF. The cut portion of EL sheet, in this example, comprised a wire that was subsequently attached to the power source.
Power Source. In order to power the ELF, for converting the ELF into an EL emitting lamp, an electric current was provided using a series of rechargeable batteries that provided DC voltage.
Inverter. A commercially available inverter (LUMX1215, AS&C CooLight, Winter Garden, Fla.) (approximately $10 U.S.) was used to power the ELF by converting approximately 14 VDC into approximately 140 VAC (100-150 VAC) at 3.5 kHz.
Filters. Inexpensive Super Gel filters (Rosco, Stamford, Conn., http://www.rosco.com/) were used for excitation and emission filters (for example, a 20″×24″ Sheet was $6.95 U.S.). In one embodiment, an excitation filter with a narrow band pass peaking at 470 nm wavelength was used for inducing fluorescence in the biological sample, see, FIG. 3a. In one embodiment, an amber excitation filter was used for filtering emission of SYBR™ Green fluorescence.
Signal detection. A standard CCD camera (Eagle Eye 2, Strategene, La Jolla, Calif.) and black & white film (FIG. 3b) was used for visualizing the SYBR™ Green fluorescence of a biological sample. Additionally, a colored photograph of a similarly prepared biological sample was produced to mimic the signal visualized by human eye (FIG. 3c).

Thus basic elements an EL base fluorescent detection device of the present invention was provided for approximately $25 U.S., excluding a CCD camera and batteries.

### Example II

A portable EL-based bench-top fluorescence detector was constructed using “off-the-shelf” relatively inexpensive components described in EXAMPLE 1 and a florescent emitting biological sample as described below.

Of the EL film from different manufacturers that were evaluated, Novatech Electro-luminescent (Blue/Green output EL lamps BG-1107, http://www.novael.com/) provided the most comprehensive specifications, for example, high brightness and moisture resistance. A blue-green base film was chosen for its higher light output than white base films, longer life expectancy, and emitted light that is similar to spectral excitation of SYBR green. Therefore for the initial evaluation of this system, a $40 sheet (20×28 cm) of EL film was purchased (Novatech Electro-luminescent (Chino, Calif.), for example, U.S. Pat. Nos. 5,667,417; 6,515,416; 6,607,413; herein incorporated by reference), then cut into the desired shape and electrically attached to an EL Lamp Driver (Inverter) (Novatech Electro-luminescent (Chino, Calif.)) that was in turn powered by rechargeable batteries (12 Duracell DC1500 2500 mah NIMH AA), as shown in a schematic diagram of an EL-Based Fluorescence Detector in FIG. 3a. A sandwich was constructed comprising a Super Gel excitation filter, biological sample i.e. post amplified products for the virulence gene ctxB from Vibrio cholera,see, below, and a Super Gel amber filter placed on top of the ELF. The ELF was turned ON, see, FIG. 3a for induced fluorescence emission from the biological sample. The emitted fluorescence was visualized with a CCD camera and photographed for providing examples of a black and white fluorescence image and colored image to represent the fluorescence as seen using a human eye.
Preparation of biological sample. A functional sample gene was amplified using conventional QPCR techniques and incorporating a SYBR Green label into the amplified double stranded product. At the completion of the real-time assay, plates comprising positive and negative samples were visualized as described above.
Virulence Gene Information. EL film was evaluated using post-amplified products for the virulence gene ctxB from Vibrio cholerae. Approximately 21.22 ng of a 237 by long amplicon was placed in each well of a multiwell plate. Organisms and virulence genes were randomly selected to demonstrate successful SYBR dye incorporation by using an IMSTAR OSA Reader™ System. An IMSTAR OSA Reader™ System was used for on-chip PCR, comprising a fluorescent microscope, a CCD camera, a temperature controlled plate holder, and image capture and analysis software. In one example a test for genes and organisms include actA gene for Listeria monocytogenes (forward primer GATTAACCCCGACATAATATTTGCA, SEQ ID NO:01, and reverse primer TGCTATTAGGTCTGCTTTGTTCGT, SEQ ID NO:02) and the ystsA for Yersinia enterocolitica (forward primer CTTCATTTGGAGCATTCGGC, SEQ ID NO:03,and reverse primer TCAGCGGTTATTGGTGTCGA, SEQ ID NO:04).

### Example III

This example shows the types of components under evaluation for use in compositions and methods of the present inventions.

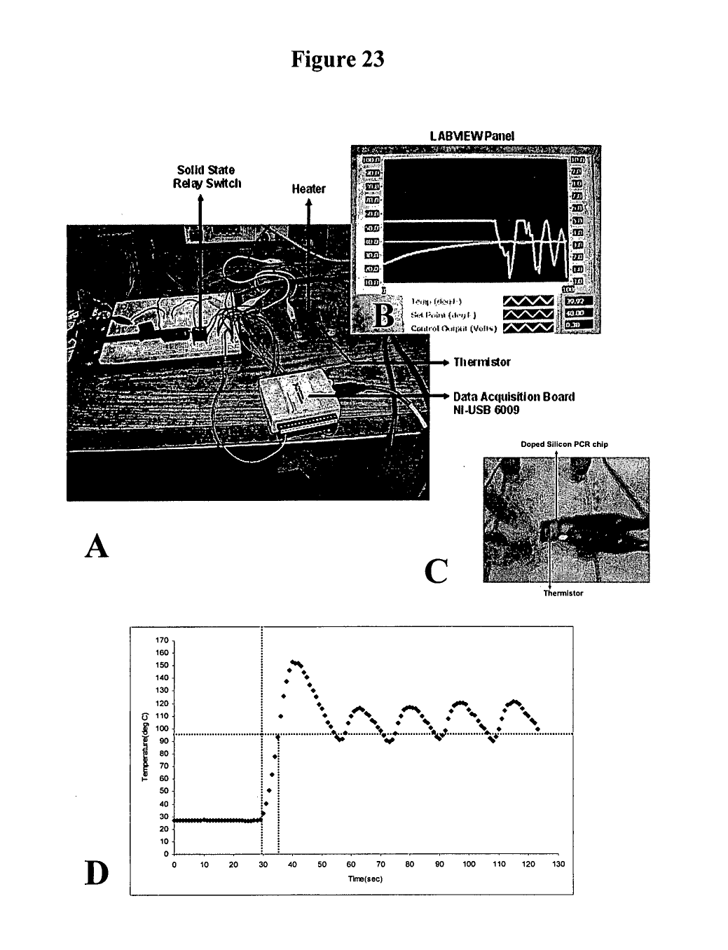

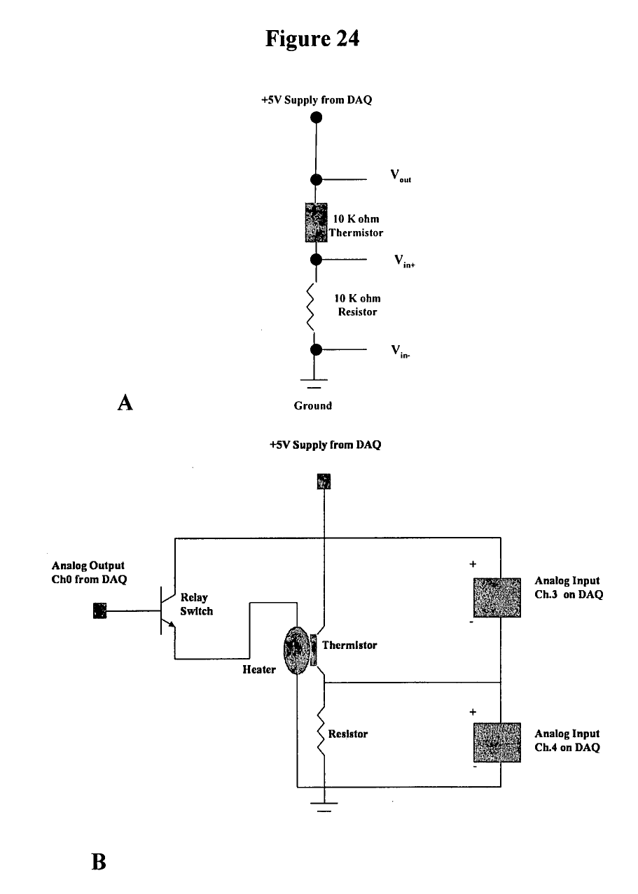

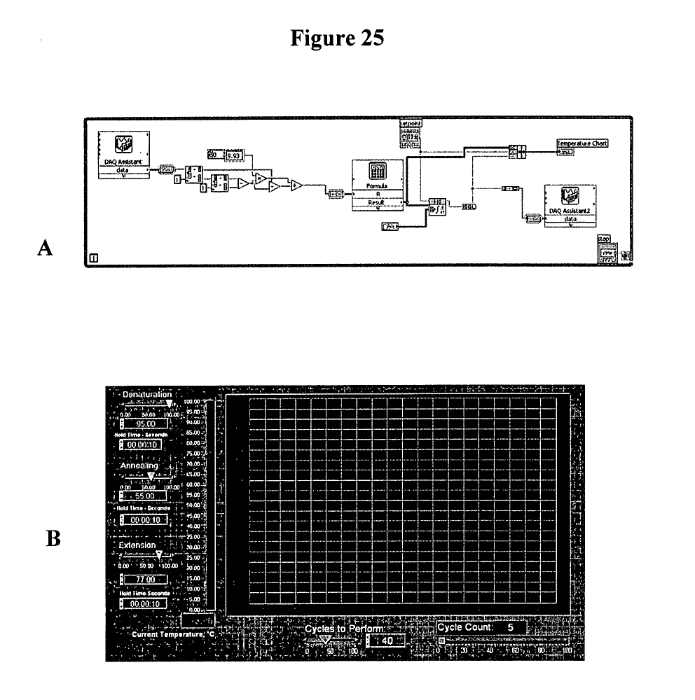

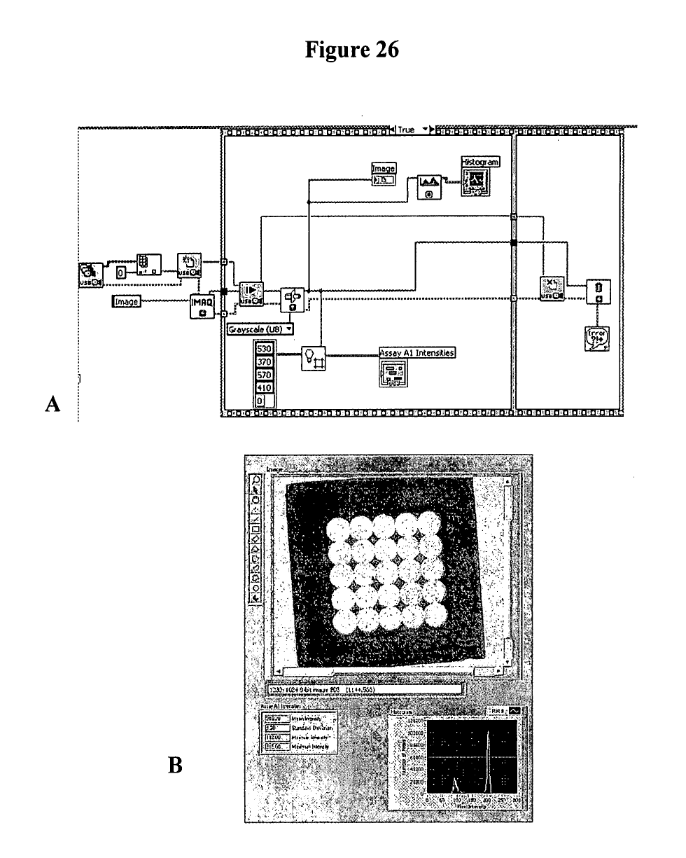

The inventors used LABVIEW for testing individual components of the present inventions, FIGS. 23-26).

This example describes developmental stages of microfluidics systems for use in detecting pathogens using PCR primers, 20 mer and 50 mer PCR oligonucleotide probes designed by the inventors. Further, this example demonstrates the use of these oligonucleotide probes in combination with microfluidic and serpentine chips (for example, see, FIG. 22) for PCR reactions, (Hashsham, et al., Microbe, Volume 2, Number 11, 2007, herein incorporated by reference).

Microfluidics-based assays were used for detecting and quantifying infectious agents by hybridizing PCR amplified products onto oligonucleotide probes. For example, the inventors developed and validated a chip (containing 8,000 microreactors, each with a diameter of 50 microns. Each reactor had oligonucleotide probes synthesized in situ using a low-cost, light-directed DNA synthesis technology. The chip was used to screen 20 different pathogens per run, based on their respective virulence and marker genes.

One of the most challenging tasks of using microfluidcs based chips with oligonucleotide probes of the present inventions was sealing of the chip after primer and sample placement inside of the chip because of the small reagent volume which evaporates even after one cycle if leaks are present. The inventors demonstrate a leakproof amplification reaction 20(a) with real time monitoring 20(b). In this experiment, the products were diffused throughout the chip with a relatively low SNR. Presence of the right size of product was confirmed by standard gel electrophoresis 20(c). A key point noted by the inventors was the appearance of the product after the 15^{th} cycle.

### Example IV

This example describes stability of freeze dried Taq polymerase and optimization of Trehalose concentrations for use in compositions and methods of the present inventions.

For field applications of a microarry (PCR) chip comprising primers and probes of the present inventions, the inventors contemplate chips with primers and reagents already dispensed in them. However, this implies that the primers/polymerase/reagents must be made stable at room temperature or even under hot climates. A common practice to obtain freeze-dried reagents is to add sugar (e.g., Trehalose) at the time of freeze-drying. Optimization of the trehalose concentration and stability of the freeze-dried reagents for long periods (6 to 12 months) are two key aspects. A trehalose concentration of 15% has generally been reported as optimal in literature and confirmed in the inventors lab (FIG. 4), although lower concentrations seem to work as well. The reagents were stable for at least one month (FIG.16).

### Example V

This example describes isothermal amplification using a helicase enzyme and primers of the present inventions for use in compositions and methods of the present inventions.

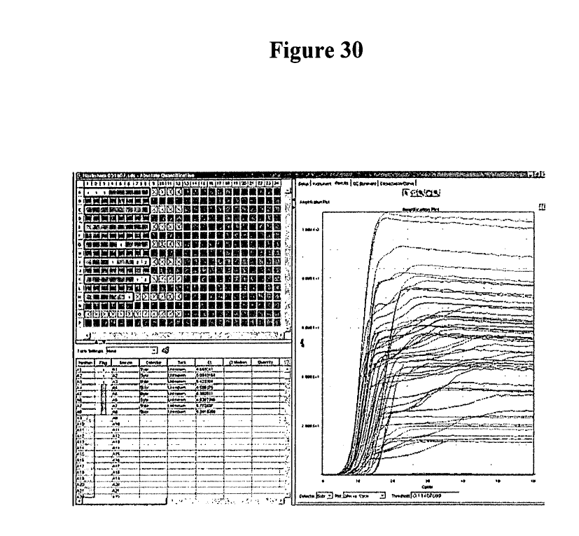

Helicase-dependent amplification is isothermal (at around 60° C.) and does not require temperature cycling. The inventors assessed the performance of this enzyme under 21 different conditions that indicated that less than 10 min. was needed for the signals to cross the background threshold. This experiment was conducted at high target concentration (˜10,000 copies). Further test are needed to evaluate the detection limit, replication, and primer design. Helicase (BioHelix Corporation, Beverly, Mass., www.biohelix.com/). (FIG. 30)

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention should not be unduly limited to such specific embodiments. Indeed, various modifications of the described modes for carrying out the invention which are obvious to those skilled in electronics, physics, medicine, microbiology, diagnostics, evolutionary biology, molecular biology or related fields are intended to be within the scope of the present invention and the following Claims.

We claim:

1. A device, comprising,
a) an electroluminescent illumination light source, wherein said electroluminescent light source comprises an electroluminescent film, and
b) a biological sample chamber.

2. The device of claim 1, wherein said electroluminescent film comprises at least one layer of indium-tin oxide.

3. The device of claim 2, wherein said layer of indium-tin oxide is optically transparent.

4. The device of claim 2, wherein said layer of indium-tin oxide is provided as a layer selected from the group consisting of a sputter deposition, an electron beam evaporation deposition, and a physical vapor deposition.

5. The device of claim 1, wherein said electroluminescent film comprises at least one layer selected from the group consisting of a polymer, a metal foil, electroluminescent phosphor ink, conductive ink, electroluminescent phosphor layer, a transparent polyester film, and a dielectric layer.

6. The device of claim 1, wherein the biological sample chamber is optically transparent.

7. The device of claim 6, wherein said biological sample chamber comprises a chip, wherein said chip is optically transparent.

8. The device of claim 7, wherein said chip selected from the group consisting of a microarray chip, a multichannel chip, and an on-chip DNA amplification chip.

9. The device of claim 7, wherein said chip comprises a biological sample.

10. The device of claim 9, wherein said biological sample comprises a fluorescent compound.

11. The device of claim 1, wherein said device further comprises at least one component selected from the group consisting of excitation filter, emission filter, optical signal detector, thin-film heater, software, a liquid crystal display, a Universal Serial Bus port, and an external case.

12. A method of detecting emitted fluorescent light, comprising:
a) providing,
i) an electroluminescent illumination light source, wherein said electroluminescent light source comprises an electroluminescent film, and
ii) a biological sample, wherein said biological sample comprises a fluorescent compound,
b) illuminating said biological sample with said electroluminescent illumination light source; and
c) detecting an optical signal emitted from said fluorescent compound.

13. The method of claim 12, wherein said electroluminescent film comprises at least one layer of indium-tin oxide.

14. The method of claim 12, wherein said biological sample is selected from the group consisting of DNA, RNA and protein.

15. The method of claim 12, wherein said biological sample comprises DNA.

16. The method of claim 15, wherein said method further comprises amplifying said DNA prior to detecting an optical signal.

17. The method of claim 15, wherein said amplifying DNA is selected from the group consisting of an isothermal amplification and a polymerase chain reaction amplification.

18. The method of claim 13, wherein said biological sample comprises a fluorescent compound, wherein said fluorescent compound is selected from the group consisting of SYBR™ Brillant Green, SYBR™ Green I, SYBR™ Green II, SYBR™ gold, SYBR™ safe, EvaGreen™, a green fluorescent protein (GFP), fluorescein, ethidium bromide (EtBr), thiazole orange (TO), oxazole yellow (YO), thiarole orange (TOTO), oxazole yellow homodimer (YOYO), oxazole yellow homodimer (YOYO-1), SYPRO® Ruby, SYPRO® Orange, Coomassie Fluor™ Orange stains, and derivatives thereof.

19. The method of claim 13, wherein said biological sample comprises a water sample.

20. The method of claim 13, wherein said detecting comprises a real-time measurement, a positive/negative answer, and pathogen identification.
